(19) United States
(12) Patent Application Publication
(10) Pub. No.: US 2024/0325007 A1
(43) Pub. Date: Oct. 3, 2024

(54) REDEPLOYABLE TISSUE RETRIEVAL BAG

(71) Applicant: Applied Medical Resources Corporation, Rancho Santa Margarita, CA (US)

(72) Inventors: Scott V. Taylor, Rancho Santa Margarita, CA (US); Adam C. Yoneda, Rancho Santa Margarita, CA (US)

(21) Appl. No.: 18/619,838

(22) Filed: Mar. 28, 2024

Related U.S. Application Data
(60) Provisional application No. 63/455,536, filed on Mar. 29, 2023.

(51) Int. Cl. A61B 17/00

(52) U.S. Cl. CPC A61B 17/00234; A61B 2017/00238; A61B 2017/00287; A61B 2017/00367

(57) ABSTRACT

A tissue retrieval system including a tissue retrieval bag deployable by an actuator from an introducer and suspended in an open configuration by support arms can be serially redeployed between a partially or fully stowed configuration and a first deployed configuration to be used in procedures to collect multiple samples. The redeployable tissue retrieval bag can include an attachment tab at an upper proximal portion to which a bead is coupled. The upper proximal portion is configured to significantly reduce stress concentrations during redeployment cycles of the tissue retrieval bag. For example, the upper proximal portion can be aligned with a proximal edge of the tissue retrieval bag. The upper proximal portion can further include a radiused profile including a relatively large radius to reduce stress concentrations.

### CROSS-REFERENCE TO RELATED APPLICATIONS

This application claims priority to and benefit of U.S. Provisional Patent Application Ser. No. 63/455,536 entitled “Redeployable Tissue Retrieval Bag” filed on Mar. 29, 2023 which is incorporated herein by reference in its entirety.

### BACKGROUND OF THE INVENTION

### Field of the Invention

The present application relates generally to apparatuses and methods for capturing and retrieving tissue from body cavities and in particular to a redeployable tissue retrieval bag for use in a specimen retrieval bag device.

### Description of the Related Art

Laparoscopic surgery is typically performed through trocars, which provide access across the abdominal wall and into the abdominal cavity. In some surgeries, tissue disposed within the abdominal cavity is cut and removed from the body. However, removal of such tissue from the body may prove difficult due to the limited confines inherent with laparoscopic surgery and the available laparoscopic surgical instruments. For example, to reduce the invasiveness to a patient, it can be desirable to introduce all of the surgical instruments through a single laparoscopic port having a relatively small size. Also, removed tissue may include an infected or cancerous mass or organ, as well as blood, bile and other liquids, all referred to herein as tissue, which may pose infection issues or other complications if left within the body.

It is desirable to grasp, capture, retain and enclose this tissue while in the body cavity, and then remove the enclosed tissue through the trocar or incision. Containment of the tissue as quickly as possible with minimal disturbance to the surgical site is also desirable. A generally compact and single unit device would also prove desirable as devices generally bulky and complicated have several shortcomings and lack optimal efficiency in particular with the limited space in operating rooms and access ports in the body cavity.

In certain procedures it can be desirable to remove multiple tissue specimens from a body cavity in a single procedure for further analysis. For example, for patients undergoing treatment for cancer, serial removal of lymph nodes is a common practice to determine the extent of the spread of the cancer or the reemergence of cancer within the patient. In order to ensure proper identification of the lymph nodes from a specific area of the body, it can be important to remove the lymph nodes in series, sometimes one at a time, from the patient to ensure an accurate determination of the stage of the cancer. Surgeons typically start with extraction of the sentinel lymph node, the lymph node closest to the tumor, and then proceed to other lymph nodes in the surrounding area. In some procedures, each lymph node that is removed from the patient is immediately analyzed for the presence of cancer by a pathologist during the surgery prior to the removal of the next lymph node to minimize the number of lymph nodes removed from the patient. Thus, it can be desirable that a tissue retrieval device be redeployable to obtain multiple tissue specimens in a single procedure.

### SUMMARY OF THE INVENTION

In certain embodiments, a tissue retrieval system is provided. The tissue retrieval system comprises a tubular introducer, an actuator, a pair of support arms, a tissue retrieval bag, and a bead. The tubular introducer has a proximal end and a distal end and defines a central longitudinal axis between the proximal end and the distal end. The tubular introducer has a lumen extending between the proximal end and the distal end. The actuator is longitudinally slidable within the lumen of the introducer. The actuator has a proximal end and a distal end. The pair of support arms extends distally along the central longitudinal axis from the distal end of the actuator. The tissue retrieval bag is removably coupled to the support arms. The tissue retrieval bag has an open end and a closed end opposite the open end. The tissue retrieval bag comprises a cuff and an upper proximal portion. The cuff extends longitudinally at the open end between a proximal end of the open end and a distal end of the open end. The cuff is configured to receive the pair of support arms. The upper proximal portion is positioned at the proximal end of the cuff and extends transversely to the longitudinal axis in a direction longitudinally proximally and away from the closed end. The bead is coupled to the upper proximal portion. The bead configured to slidably receive the pair of support arms.

In certain embodiments, a tissue retrieval system is provided herein. The tissue retrieval system comprises a tubular introducer, an actuator, a pair of support arms, a tissue retrieval bag, and a bead. The tubular introducer has a proximal end and a distal end and defines a central longitudinal axis between the proximal end and the distal end. The tubular introducer has a lumen extending between the proximal end and the distal end. The actuator is longitudinally slidable within the lumen of the introducer. The actuator has a proximal end and a distal end. A pair of support arms extends distally along the central longitudinal axis from the distal end of the actuator. The tissue retrieval bag is removably coupled to the support arms. The tissue retrieval bag has an open end and a closed end opposite the open end and defines a depth axis extending from the open end to the closed end orthogonally to the central longitudinal axis. The open end extends along the central longitudinal axis from a proximal end to a distal end. The tissue retrieval bag comprises a distal edge, a proximal edge, and an upper proximal portion. The distal edge extends from the distal end of the open end to the closed end. The proximal edge extends from the proximal end of the open end to the closed end. The proximal edge extends transversely to the central longitudinal axis. The upper proximal portion is positioned at the proximal end of the open end and extends in a direction colinear with the proximal edge away from the closed end with respect to the depth axis. The bead is coupled to the upper proximal portion, the bead configured to slidably receive the pair of support arms.

In certain embodiments, a tissue retrieval bag for use in a redeployable tissue retrieval system is provided herein. The tissue retrieval bag comprises an open end, a closed end, a distal edge, a proximal edge, an attachment tab, and a bead. The open end extends along a central longitudinal axis from a proximal end of the open end to a distal end of the open end. The closed end is opposite the open end and defines a depth axis extending from the open end to the closed end orthogonally to the central longitudinal axis. The distal edge extends from the distal end of the open end to the closed end. The proximal edge extends from the proximal end of the open end to the closed end. The proximal edge extends transversely to the central longitudinal axis. The attachment tab is positioned at the proximal end of the open end and extends in a direction colinear with the proximal edge away from the closed end with respect to the depth axis. The bead is coupled to the attachment tab.

### BRIEF DESCRIPTION OF THE DRAWINGS

FIG. 1 is a perspective view of an embodiment of tissue retrieval system with an embodiment of tissue retrieval bag in a first deployed configuration;

FIG. 2 is a side view of the tissue retrieval system of FIG. 1;

FIG. 3 is a perspective view of an embodiment of tissue retrieval system with an embodiment of tissue retrieval bag in a stowed configuration;

FIG. 4 is a top view of the tissue retrieval system of FIG. 1 positioned through an access port in a surgical site with the tissue retrieval bag in a deployed configuration;

FIG. 5 is a top view of the tissue retrieval system of FIG. 1 positioned through an access port in a surgical site with the tissue retrieval bag in a redeployable closed configuration;

FIG. 6 is a top view of the tissue retrieval system of FIG. 1 positioned through an access port in a surgical site with the tissue retrieval bag in a fully deployed configuration;

FIG. 7 is a top view of the tissue retrieval system of FIG. 1 positioned through an access port in a surgical site with the tissue retrieval bag in a fully deployed cinched configuration;

FIG. 8 is a side view of an embodiment of tissue retrieval bag and bead assembly of the tissue retrieval system of FIG. 1;

FIG. 9 is a side view of an embodiment of tissue retrieval bag of the assembly of FIG. 8;

FIG. 10 is a side view of an embodiment of prior art tissue retrieval bag and bead assembly;

FIG. 11 is a side view of an embodiment of prior art tissue retrieval bag of the assembly of FIG. 10;

FIG. 12 is a schematic view of a sheet of material cut according to a die cut pattern for the tissue retrieval bag of FIG. 9;

FIG. 13 is a schematic view of a partially formed tissue retrieval bag formed according to the die cut pattern of FIG. 12;

FIG. 14 is a schematic view of a sheet of material cut according to a die cut pattern for another embodiment of tissue retrieval bag;

FIG. 15 is a schematic view of a partially formed tissue retrieval bag formed according to the die cut pattern of FIG. 14;

FIG. 16 is a schematic view of a sheet of material cut according to a die cut pattern for another embodiment of tissue retrieval bag;

FIG. 17 is a schematic view of a partially formed tissue retrieval bag formed according to the die cut pattern of FIG. 16;

FIG. 18 is a schematic view of a sheet of material cut according to a die cut pattern for another embodiment of tissue retrieval bag;

FIG. 19 is a schematic view of a partially formed tissue retrieval bag formed according to the die cut pattern of FIG. 18;

FIG. 20 is a side view of an embodiment of another embodiment of tissue retrieval bag;

FIG. 21 is a schematic view of a sheet of material cut according to a die cut pattern for an embodiment of tissue retrieval bag;

FIG. 22 is a schematic view of a partially formed tissue retrieval bag formed according to the die cut pattern of FIG. 21;

FIG. 23 is a side view of the embodiment of tissue retrieval bag formed according to the die cut pattern of FIG. 21;

FIG. 24 is a side view of the embodiment of tissue retrieval bag and bead assembly of formed according to the die cut pattern of FIG. 21;

FIG. 25 is a schematic view of a sheet of material cut according to a die cut pattern for an embodiment of tissue retrieval bag;

FIG. 26 is a schematic view of a reinforcement tab for use with the die cut pattern of FIG. 25;

FIG. 27 is a schematic view of a partially formed tissue retrieval bag formed according to the die cut pattern of FIG. 25 and the reinforcement tab of FIG. 26;

FIG. 28 is a schematic view of a partially formed tissue retrieval bag formed according to the die cut pattern of FIG. 25 and the reinforcement tab of FIG. 26 having a formed cuff and a cord loop;

FIG. 29 is a side view of the embodiment of tissue retrieval bag formed according to the die cut pattern of FIG. 25 and the reinforcement tab of FIG. 26;

FIG. 30 is a side view of the embodiment of tissue retrieval bag and bead assembly formed according to the die cut pattern of FIG. 25 and the reinforcement tab of FIG. 26;

FIG. 31 is a schematic view of a sheet of material cut according to a die cut pattern for an embodiment of tissue retrieval bag;

FIG. 32 is a schematic view of a partially formed tissue retrieval bag formed according to the die cut pattern of FIG. 31;

FIG. 33 is a side view of the embodiment of tissue retrieval bag formed according to the die cut pattern of FIG. 31;

FIG. 34 is a side view of the embodiment of tissue retrieval bag and bead assembly formed according to the die cut pattern of FIG. 31;

FIG. 35 is a schematic view of a sheet of material cut according to a die cut pattern for an embodiment of tissue retrieval bag;

FIG. 36 is a schematic view of a reinforcement ring for use with the die cut pattern of FIG. 35;

FIG. 37 is a schematic view of a partially formed tissue retrieval bag formed according to the die cut pattern of FIG. 35 and the reinforcement ring of FIG. 36;

FIG. 38 is a schematic view of a partially formed tissue retrieval bag formed according to the die cut pattern of FIG. 35 and the reinforcement ring of FIG. 36 having a formed cuff and a cord loop;

FIG. 39 is a side view of the embodiment of tissue retrieval bag formed according to the die cut pattern of FIG. 35 and the reinforcement ring of FIG. 36;

FIG. 40 is a side view of the embodiment of tissue retrieval bag and bead assembly formed according to the die cut pattern of FIG. 35 and the reinforcement ring of FIG. 36;

FIG. 41 is a schematic view of a partially formed tissue retrieval bag formed according to the die cut pattern of FIG. 12 and a reinforcement disk positioned thereon; and

FIG. 42 is a side view of the embodiment of tissue retrieval bag formed according to the die cut pattern of FIG. 41 and the reinforcement disk of FIG. 41.

### DETAILED DESCRIPTION OF THE INVENTION

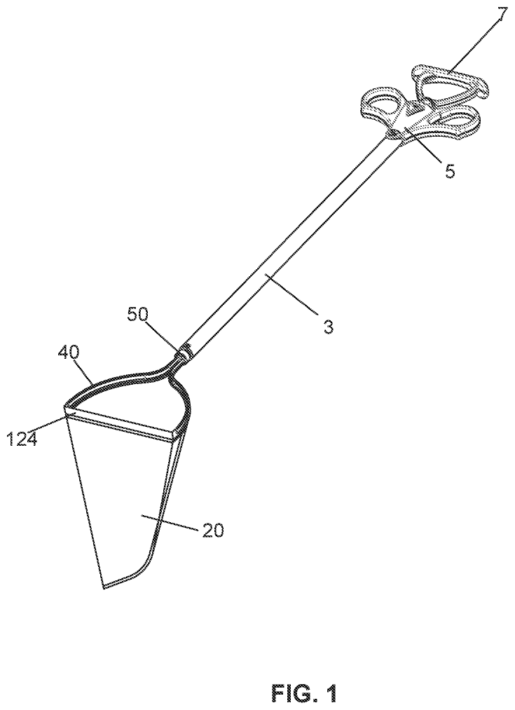

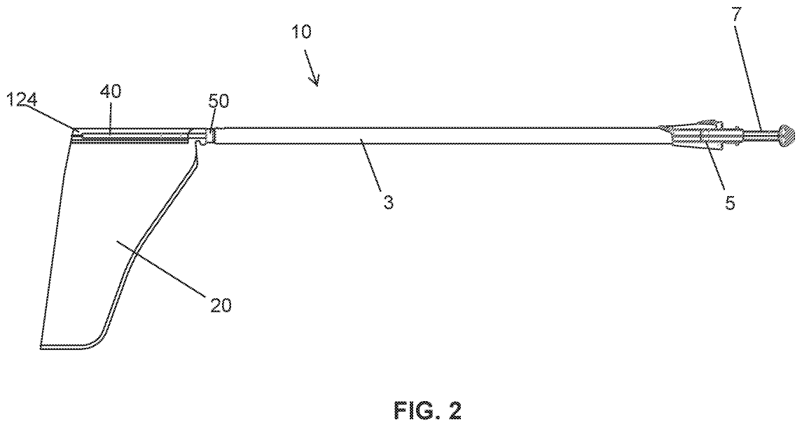

With reference to FIGS. 1 and 2, an embodiment of tissue retrieval system 10 is illustrated. The illustrated tissue retrieval system can be used for containing and withdrawing excised tissue specimens from within a body cavity. Certain aspects of tissue retrieval systems are described in U.S. Pat. No. 11,547,428, entitled “REDEPLOYABLE TISSUE RETRIEVAL SYSTEM,” U.S. Pat. No. 8,721,658, entitled “TISSUE RETRIEVAL SYSTEM,” and U.S. Pat. No. 9,033,995, entitled “SINGLE INCISION LAPAROSCOPIC TISSUE RETRIEVAL SYSTEM.” Each of these patents is incorporated by reference herein in its entirety.

In various embodiments, tissue retrieval systems 10 described herein provide a versatile redeployable retrieval system that can be used for serially containing and withdrawing multiple small tissue specimens during a surgical procedure without removing the retrieval bag from the device while also enabling the extraction of a large tissue specimen by cinching the retrieval bag and then detaching the retrieval bag from the introducer. In some procedures, the surgeon may only need the ability to serially contain and withdraw small specimens from the patient. In some procedures, the surgeon may need the ability to serially contain and withdraw multiple small tissue specimens and may then also need to contain and extract a large specimen. In other procedures, the surgeon may only need to contain and extract a large tissue specimen. As the retrieval systems described herein meet many different surgical requirements, a hospital or surgery center using these systems may be able to limit the number of retrieval system models to stock for its surgical needs.

The illustrated tissue retrieval system can desirably be used in certain procedures for serially containing and withdrawing multiple excised small tissue specimens. Advantageously, certain embodiments of tissue retrieval system described herein can permit the use of a single retrieval system for the serial removal of multiple lymph nodes during a procedure.

With reference to FIGS. 1 and 2, in certain embodiments, a tissue retrieval system 10 includes a retrieval bag 20 at its distal end, which provides a receptacle for tissue specimens. The retrieval bag 20 can have an open end and a closed end opposite the open end. The retrieval bag 20 can further comprise a cuff 124 formed at the open end for receiving a pair of support arms 40 to suspend the retrieval bag 20 and a cord loop to selectively cinch the open end of the retrieval bag. In various embodiments, the retrieval bag can be formed of a ripstop nylon and polyurethane laminate, polyurethane, or a combination of both materials. For example, the retrieval bag can be formed from a ripstop nylon and polyurethane laminate and can include a polyurethane reinforcement at the tip of the retrieval bag to increase the burst strength and puncture resistance of the retrieval bag. The reinforcement can be comprised of a ripstop nylon or a ripstop nylon and polyurethane laminate. As an alternative to the ripstop nylon and polyurethane laminate, the ripstop nylon can be coated with polyurethane.

In a procedure to retrieve multiple small specimens, after insertion of a tissue specimen into the retrieval bag, the retrieval bag 20 can be reversibly closed to prevent spillage of its contents and to prevent contamination of the body cavity and body cavity wall during withdrawal of the retrieval bag 20 from within the body cavity. Once the retrieval bag is withdrawn from the body cavity, the retrieval bag is reopened, the tissue specimen can be removed, labeled, and then forwarded to a pathologist for analysis. The retrieval bag can then be fully retracted into the introducer tube and redeployed within the patient for subsequent containment and withdrawal of the next targeted tissue specimen.

The tissue retrieval system 10 can also be used in procedures for removing large tissue specimens, such as a gallbladder or kidney, from within a body cavity. In these procedures, the retrieval bag 20 can be fully cinched closed and detached from the system for subsequent withdrawal of the retrieval bag 20 through the body wall. In some procedures, such as those related to the excision of cancerous tissue, it may be desirable to first serially remove small tissue specimens, such as lymph nodes, and to then remove the larger tissue specimen. In this case, the tissue retrieval system 10 enables the retrieval bag to be reversibly closed for serially removing smaller tissue specimens and enables the retrieval bag to be fully cinched closed and detached for removal of the large tissue specimen.

With reference to FIGS. 1-7, certain embodiments of tissue retrieval system 10 are illustrated in various configurations for use in procedures to retrieve multiple tissue specimens in a single procedure. With reference to FIGS. 1 and 2, the tissue retrieval system 10 is illustrated in a first deployed condition. In the illustrated embodiment, the tissue retrieval system has an introducer 3 and an actuator 7 or actuation rod. The introducer 3 in one aspect has a tubular configuration with a hollow lumen and a handle assembly 5 extending from a proximal end of the introducer 3. In some embodiments, the introducer 3 can be sized and configured for placement through a standard-size trocar. For example, it can be desirable that the introducer 3 can be sized as a 5 mm laparoscopic surgical instrument to be introduced through relatively small diameter trocars such as 5-7 mm trocars. In other embodiments, the introducer 3 can be sized as a 10 mm or a 12 mm laparoscopic surgical instrument. In some embodiments, the introducer 3 can have a non-standard size for application at a specific location. In some embodiments, the tissue retrieval system 10 can include a relatively long introducer, such as, for example, a 45 cm long introducer 3 to improve access to the surgical site.

The handle assembly 5 of the illustrated embodiment can comprise a compact handle member that can be adapted for placement adjacent other surgical instruments in a single port laparoscopic surgical site. Thus, in some embodiments, the tissue retrieval system is adapted to be utilized during single incision laparoscopic procedures. In other embodiments, the handle assembly can include a pair of finger loops or grips formed with or otherwise coupled to the handle assembly 5 that can be utilized to hold or stabilize the introducer 3 as desired.

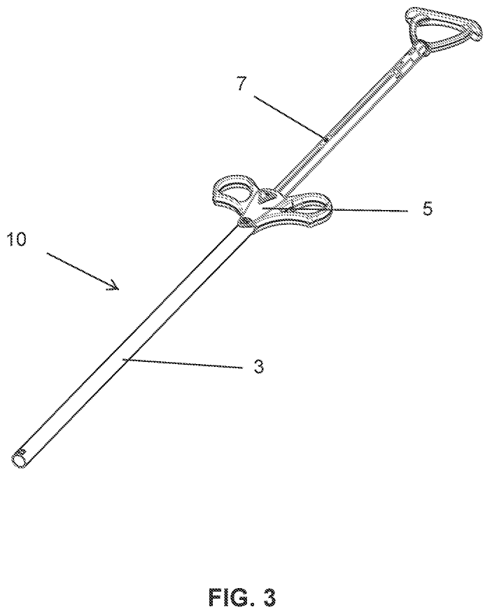

In the illustrated embodiment of tissue retrieval system 10, the introducer 3 has a proximal end and a distal end that are generally open, which can facilitate access to the hollow lumen. As illustrated, the actuator 7 extends into the hollow lumen from the open proximal end thereof, and at least a portion of the actuator 7 is slidably movable within the hollow lumen of the introducer 3. With reference to FIG. 2, the actuator rod can be withdrawn to a proximal position for insertion to a surgical site through an access port such as a trocar. With the actuator 7 in the proximal position the tissue retrieval bag 20 is in a stowed configuration positioned in the hollow lumen of the introducer 3. The actuator 7 in one aspect has a handle such as a thumb loop extending from a proximal end thereof. The handle provides a graspable portion of the device to control or facilitate movement of the actuator 7 relative to the introducer 3 between the proximal position (illustrated in FIG. 3), and a first deployed condition of the tissue retrieval system (illustrated in FIG. 1).

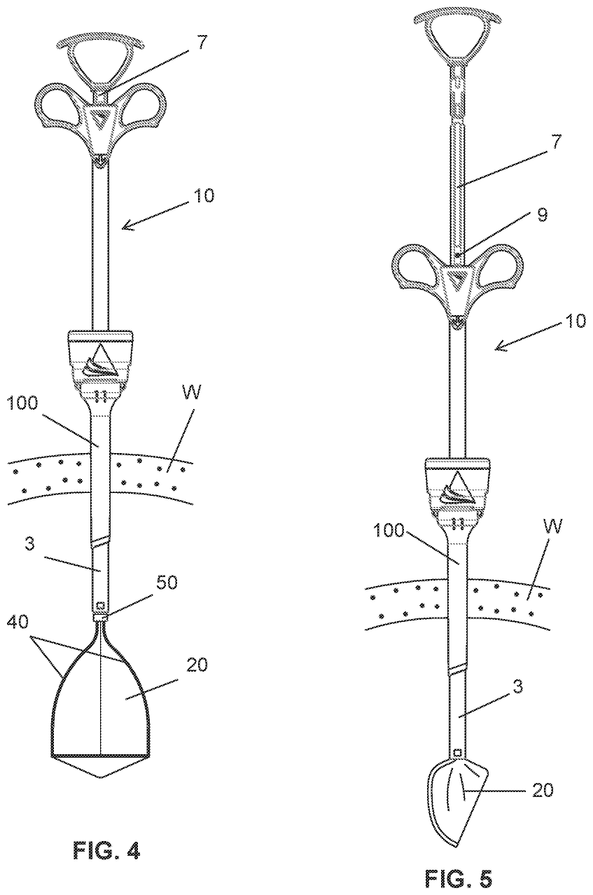

With reference to FIGS. 4-7 exemplary methods for using the illustrated embodiment of tissue retrieval system 10 to retrieve tissue specimens at a surgical site are illustrated. In certain embodiments, the tissue retrieval system can be provided in either a stowed configuration (FIG. 3) or a first deployed configuration (FIG. 1). In some embodiments, it can be desirable that the tissue retrieval system 10 is packaged with the retrieval bag 20 deployed out of the distal end of the introducer tube to ensure that the retrieval bag 20 maintains its original configuration and easily unfurls during surgical use. During clinical use, an access device such as a trocar 100 comprising a trocar cannula and trocar seal housing is first placed through a body wall W leaving the trocar cannula disposed across the body wall W. If the tissue retrieval system 10 was provided in a first deployed configuration, a nurse or surgeon configures the tissue retrieval system to a stowed configuration (FIG. 3) by fully retracting the retrieval bag 20 within the introducer 3 tube by withdrawing the actuator 7 in a proximal direction to position the actuator rod in a proximal position. The tissue retrieval system 10 can then be inserted into the trocar seal housing and trocar cannula until the distal end of the introducer 3 tube extends beyond the distal end of the trocar cannula. The retrieval bag 20 is then deployed from within the introducer 3 tube and into the body cavity by advancing the actuator in a distal direction to the first deployed position (FIG. 3).

With continued reference to FIG. 4, once extended into the body cavity, the retrieval bag 20 in the first deployed configuration is suspended and held open by two support arms 40 that extend into a cuff at the open end of the retrieval bag. In the illustrated embodiment, the retrieval bag includes a bead 50 attached to a proximal portion of the cuff through which the support arms 40 extend. In the first deployed position, the illustrated embodiment of tissue retrieval system comprises a retaining latch coupled to a distal end of the actuator 7 and engaged with the bead 50. Advantageously, the retaining latch enables the surgeon to selectively control a release of the retrieval bag 20 from the support arms and the actuator. While in the first deployed position, the retaining latch is engaged with the bead 50 to prevent the release of the retrieval bag 20 relative to the support arms, the retaining latch is releasably attached to the bead 50. When engaged with the bead 50, the retaining latch enables the retrieval bag 20 to be completely retracted into the introducer 3 (FIG. 3) with the actuator 7 withdrawn to a proximal position for subsequent insertion through the trocar. When engaged with the bead 50, the retaining latch also enables the retrieval bag 20 to be partially retracted into the introducer tube (FIG. 5) to a redeployable closed configuration, allowing reversible closing of the opening of the retrieval bag. Thus sequential, repeated opening and closing of the tissue retrieval bag 20 can be used to contain multiple small tissue specimens in a single procedure for subsequent withdrawal from the patient achieved by repeatedly advancing the actuator to a first deployed position (FIG. 4) and withdrawing the actuator towards the proximal position (FIG. 5) to position the tissue retrieval bag in a redeployable closed configuration.

With reference to FIG. 5, once a small tissue specimen has been positioned in the retrieval bag, partial retraction and closure of the retrieval bag 20 is achieved by retracting the actuator 7 into the introducer 3 tube until the support arms 40 and the cuff are drawn into the introducer 3 tube, leaving a distal portion of the retrieval bag 20 with contained tissue outside of the introducer 3 tube. In certain embodiments, the actuator can comprise visual indicia such as dot 9. The visual indicia can be positioned at a location on the actuator that is exposed at a proximal end of the introducer 3 tube when the retrieval bag 20 has been closed. In certain embodiments, the dot 9 is pad printed and is an indicator for the surgeon to indicate that the actuator has been sufficiently retracted to close the bag. Various techniques can then be used to remove the tissue specimen from the surgical site across the body wall. For example, the tissue retrieval system 10 can be withdrawn through the trocar, leaving the trocar disposed across the body wall, or the tissue retrieval system can be directly withdrawn through the body wall after withdrawal of the trocar from the patient. For very small tissue specimens, the entire retrieval bag 20 with the contained specimen can be withdrawn into the introducer tube. In this case, the tissue retrieval system can be withdrawn through the trocar, leaving the trocar disposed across the body wall.

Once the tissue retrieval system 10 has been withdrawn from the surgical site, the actuator 7 can be advanced distally to the first deployed position (FIG. 1) and the tissue specimen removed for analysis. If further small tissue specimens are desired to be obtained, the actuator 7 can then be withdrawn to the proximal position (FIG. 3) for reintroduction to the surgical site as described above. In certain embodiments, the tissue retrieval system can include stop mechanisms to limit travel of the actuator between the proximal position and the first deployed position until it is desired to separate the tissue retrieval bag 20 from the actuator 7. Advantageously, these stop mechanisms can reduce the potential for an inadvertent full deployment of the tissue retrieval bag before all of the desired tissue specimens have been collected for analysis.

The tissue retrieval system 10 can also be used in procedures to extract relatively large tissue specimens such as a gallbladder, appendix, or kidney. In these procedures, desirably with the tissue retrieval bag in the first deployed configuration (FIG. 4) during placement of a large tissue specimen such as the gallbladder, the retaining latch prevents inadvertent movement of the retrieval bag relative to the support arms and the actuator. Once a large specimen has been positioned in the tissue retrieval bag, it is desirable to cinch and fully detach the retrieval bag from the introducer, leaving the cinched retrieval bag within the body cavity. If the procedure requires the serial removal of small tissue specimens such as lymph nodes and then requires the removal of a large tissue specimen, the cinching and detachment of the retrieval bag would be completed after the serial removal of the smaller tissue specimens. Once the retrieval bag is cinched closed and detached from the introducer, the surgeon then removes the trocar from the body wall and can then extract the retrieval bag through the body wall.

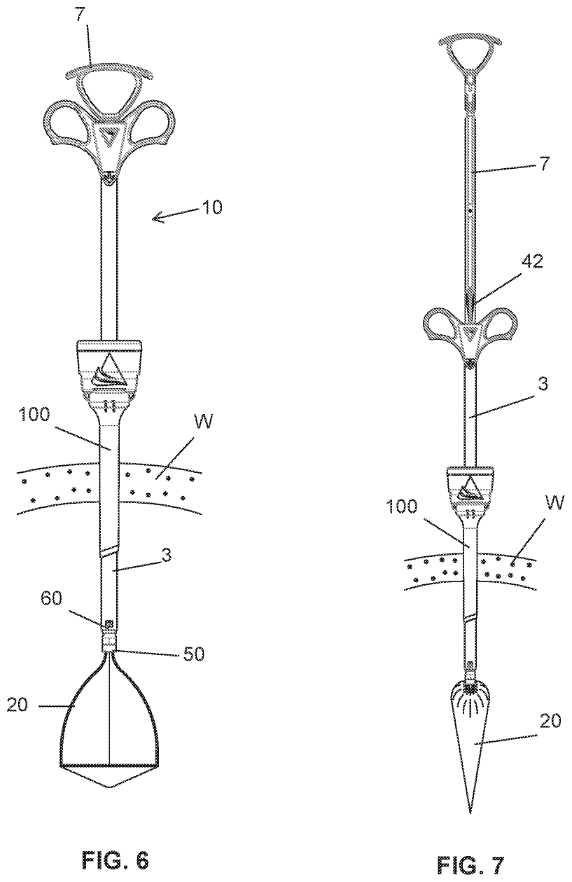

In certain embodiments, the tissue retrieval system 10 can be selectively actuated from the first deployed configuration (FIG. 4) to position the actuator and tissue retrieval bag in a fully deployed configuration (FIG. 6). Once a surgeon determines that it is desirable to separate the tissue retrieval bag from the introducer, such as when a large or final tissue specimen is placed within the retrieval bag, the surgeon actuates a deployment release mechanism for example by pressing a deployment release button on the handle assembly. With actuation of the deployment release mechanism, the actuator can be advanced distally beyond the first deployed position to a second or fully deployed position (FIG. 6). Advancement of the actuator to its fully deployed position causes the bead and the distal end of the retaining latch to be disposed out of the distal end of the introducer tube.

With reference to FIGS. 6 and 7, in certain embodiments, the tissue retrieval system 10 can include a stop mechanism to prevent reintroduction of the bead into the distal end of the introducer 3 once the actuator 7 has been advanced to the fully deployed position. For example, in the illustrated embodiment, the tissue retrieval system 10 comprises a bead stop 60, which is positioned between the actuator 7 and the bead 50. With the actuator in the fully deployed position, the bead stop is also advanced to its most distal position where it lockingly engages the introducer to obstruct the distal end of the introducer 3. This obstruction prevents the bead and the retrieval bag, once fully deployed, from being withdrawn into the introducer tube. This obstruction also presents a bearing surface such that subsequent proximal withdrawal of the actuator from the fully deployed position withdraws the support arms from the cuff of the tissue retrieval bag 20 and cinches the tissue retrieval bag 20 (FIG. 7). Once the retrieval bag is cinched closed, a small loop of the cord loop 42 is exposed near the proximal end of the introducer 3 tube.

In certain embodiments, the tissue retrieval system comprises a cord loop 42 positioned to selectively cinch the opening of the tissue retrieval bag into a cinched configuration. The cord loop 42 can extend through the cuff at the open end of the tissue retrieval bag 20 and extend proximally into the introducer along a receiving channel in the actuator. The cord loop 42 can be removably coupled to the actuator. In certain embodiments, the cord loop is dimensioned such that its proximal end is positioned between the proximal end and the distal end of the actuator and retained by the actuator. Desirably, in certain embodiments, the cord loop 42 is dimensioned such that its length enables cinching of the retrieval bag and exposure of the cord loop 42 on the actuator after cinching. When the retrieval bag is cinched closed, the cord loop 42 is fully tensioned. Prior to full tensioning of the cord loop 42 during cinching of the retrieval bag, the cord loop 42 is fully contained within the introducer tube of the retrieval system. In certain embodiments, the cord loop is not exposed during deployment of the retrieval bag or during retraction of the retrieval bag within the introducer tube. For example, during insertion to the surgical site and serial containment and withdrawal of multiple small tissue specimens (FIGS. 3-5), the cord loop is not exposed. In these embodiments, the cord loop 42 is only exposed to the surgeon when the cord loop is fully tensioned and the retrieval bag is cinched closed (FIG. 7). This feature prevents the surgeon from unintentionally grasping, cutting, releasing, or tensioning the cord loop during use of the device. The cord loop can be stored in the introducer tube in a non-tensioned condition with a portion of the cord loop being folded and stored in a receiving channel on the underside of the actuator.

The cord loop 42 can provide an ergonomic and high strength means for grasping and withdrawing the retrieval bag through the abdominal wall. As the cord is formed of a single loop of cord, there is no limitation in terms of being able to manually grip the cord loop. The cord loop also provides a robust means for withdrawing the retrieval bag through the body wall as two strands of the cord absorb the tension being applied by the surgeon. For example, if the surgeon is applying a tensile force of 20 lbs. to the cord loop during withdrawal of the retrieval bag, each strand of the cord is being tensioned at a force of 10 lbs. Certain other retrieval systems have included relatively small loops of material which can make it difficult to manually grasp the loop. Other retrieval systems have also included a small loop that is connected to a single strand of cord or line where a 20 lb. tensile force applied by the surgeon results in a 20 lb. tensile force being applied to the cord. In some cases, the single strand of cord or line of these other retrieval systems can fail during withdrawal of the retrieval bag.

Another advantage provided by the cord loop 42 is that the cord loop 42 is relatively short after cinching of the retrieval bag, providing for easier management of the cord and easier withdrawal of the retrieval bag through the body wall. In certain embodiments of tissue retrieval systems described herein, the length of the cord loop with the retrieval bag cinched is about 14″. Other retrieval systems can have a cord with a length greater than about 25″ after cinching of the retrieval bag making the cord unwieldy and difficult to manage and use during withdrawal of the retrieval bag through the body wall.

With reference to FIG. 7, once the tissue retrieval bag 20 has been cinched, there are at least two techniques for withdrawal of the cinched retrieval bag from within the body cavity. For the first method, the retrieval bag can be completely detached and removed from the actuator and introducer tube by lifting the cord loop 42 from the retaining slot on the actuator.

With reference to FIG. 7, once the cord loop has been decoupled from the actuator, the device and the trocar seal and cannula can then be withdrawn from the body wall leaving the retrieval bag 20 in the body cavity and the cord loop 42 disposed across the body wall. The neck of the retrieval bag can then be withdrawn through the body wall using the bead as a dilator to aid with movement of the retrieval bag through the layers of tissue fibers in the body wall. Once the neck of the retrieval bag has traversed the body wall, the retrieval bag can then be reopened by manually grasping the closed end of the retrieval bag and the bead and sliding the bead along the cord. The retrieval bag can then be accessed to remove or compact its contents to aid with complete withdrawal from the body cavity using standard open and laparoscopic instrumentation such as forceps, graspers, and aspiration probes. Once the bulk of the contents are removed, the retrieval bag can then be closed by manually grasping the open end of the retrieval bag and the bead and sliding the bead along the cord. The cord loop can then be grasped manually and the retrieval bag then completely withdrawn from the body cavity. In some procedures, the surgeon may withdraw the retrieval bag through the body wall without reopening the retrieval bag.

A second method for withdrawal of the retrieval bag 20 from within the body cavity can be used for smaller tissue specimens, such as a small gallbladder, placed in the retrieval bag which are not likely to need to be aspirated, compacted, or removed from the retrieval bag prior to withdrawal of the retrieval bag through the body wall. In this case, the cord loop can be left attached to the actuator and the entire device along with the trocar seal and cannula can be simultaneously withdrawn from the body cavity and through the body wall.

In certain embodiments, the bead 50 comprises a pair of channels through which the support arms can slide. The bead 50 can further comprise a hole through which the cord loop runs that frictionally engages the cord loop to enable the retrieval bag to be cinched closed and reopened as needed once the retrieval bag has been completely detached from the actuator and the introducer. The bead can also be configured to releasably engage the retaining latch. In the certain embodiment, the bead 50 includes a recess and a ledge positioned in the recess to engage a distal end of the retaining latch. In certain embodiments, the bead 50 is injection molded from polycarbonate, and, in other embodiments, the bead 50 can be formed from other materials such as nylon, ABS, and polyester.

With continued reference to FIG. 7, in certain embodiments, the bead 50 is configured to facilitate withdrawal of the cinched retrieval bag through the body wall. In certain embodiments a proximal end of the bead 50 can comprise a blunt, tapered, or radiused surface to facilitate withdrawal of the bead through a body wall. Thus, the bead can dilate muscle and tissue fibers of the body wall during withdrawal of the retrieval bag. Advantageously, as the bead traverses the body wall, the cinched cuff and the remainder of the retrieval bag can easily follow. Retrieval systems that do not include a bead can require the bunched cuff of the cinched retrieval bag to be pulled directly into the body wall where the bunched cuff can sometimes catch on the body wall resulting in an increase in the extraction force during withdrawal of the retrieval bag.

In certain embodiments, the bead 50 includes a passage, bore, or hole through which the cord loop runs that frictionally engages the cord loop to enable the retrieval bag to be cinched closed and reopened as needed once the retrieval bag has been detached from the introducer. Thus, during withdrawal of the retrieval bag from the body cavity, once the neck of the retrieval bag has traversed the body wall, the retrieval bag can advantageously be easily reopened to enable access to remove or compact its contents. Previous retrieval bags with no bead can be very difficult or impossible to reopen once the retrieval bag has been cinched closed.

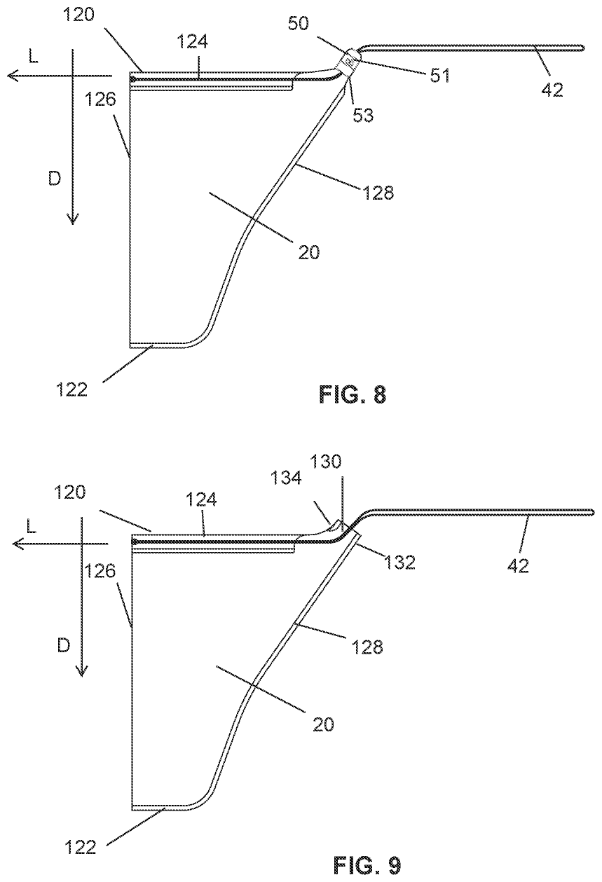

With reference to FIG. 8, in certain embodiments, the bead 50 can comprise a two-piece assembly comprising a bead body 51 and an annular clamp 53 positioned around a portion of the bead body. A portion of the tissue retrieval bag can be positioned between the bead body 51 and the clamp 53 to couple the tissue retrieval bag to the bead 50. Various joining techniques such as gluing with chemical adhesives, ultrasonic welding, heat sealing, or other chemical, thermal, or mechanical joining processes can further secure the retrieval bag to the bead body and/or clamp. In other embodiments, the bead can be a single component without an encircling clamp, and the single component bead can be coupled to the retrieval bag with various chemical, thermal, or mechanical joining processes.

As opposed to tissue retrieval systems configured for a single deployment, it is desirable that tissue bags 20 for use in redeployable tissue retrieval systems 10 are configured to resist tearing or rupture due to material fatigue or relatively high loads that can be encountered over multiple tensile loading cycles with the tissue retrieval bag 20 maintained coupled to the introducer. During withdrawal of the introducer 3 with a closed retrieval bag 20 by the surgeon through the trocar or alternatively, directly through the incision, a relatively high tensile force can be applied through the retaining latch to the bead 50 and through a proximal portion of the retrieval bag 20 where the bead 50 is attached. The tensile force typically increases as the distal portion of the bag 20 that contains the specimen contacts either the distal end of the trocar or the anterior side of the incision. A large tissue specimen tends to induce a higher tensile force versus a smaller tissue specimen. High tensile forces will generate correspondingly high stresses in a proximal section of the retrieval bag 20, particularly where the bead 50 is attached to the retrieval bag 20 and portions of the retrieval bag 20 extending directly beyond the bead 50.

When the retrieval bag 20 is partially retracted into the introducer 3 tube, the opening of the bag 20 is closed and drawn into the introducer 3 tube. As the retrieval system is retracted, the portion of the retrieval bag 20 that contains the specimen is drawn into the distal end of the trocar or drawn into the incision. In this configuration, a tensile force is applied to the retrieval bag 20 along a central longitudinal axis of the introducer 3 tube since the bead 50, the portion of the retrieval bag 20 that the bead is coupled to, and the cuff of the retrieval bag are positioned within the introducer 3 tube. This longitudinal tensile force translates into a tensile force that runs through the portion of the retrieval bag 20 where the bead 50 is attached and then along a length of the retrieval bag 20 between the where the bead 50 is attached and where the specimen is located at the distal tip of the retrieval bag 20. During typical surgical use, almost all of the stress that results in the retrieval bag beyond where the bead 50 is attached runs along a proximal portion of the retrieval bag 20 with minimal stress running through the cuff portion of the retrieval bag 20.

The distribution of stress when the retrieval bag 20 is in the closed configuration for the withdrawal of small tissue specimens such as lymph nodes can be markedly different from the distribution of stress when the retrieval bag 20 is in the cinched configuration for the withdrawal of large tissue specimens such as a gallbladder. When the retrieval bag 20 is cinched closed and detached from the actuator 7, the surgeon will apply a tensile force to the cord loop 42. This tensile force then translates through the cuff and is evenly distributed across a width of the retrieval bag 20 between the cuff and the distal tip of the bag that contains the specimen. In this case, there is substantially no tensile force running through the bead 50, through the portion of the retrieval bag 20 that the bead is attached to, or through the portions of the retrieval bag 20 extending directly beyond the bead. The stress induced in the cinched retrieval bag 20 is therefore evenly distributed within the cuff and throughout the width of the retrieval bag 20 between the cuff and the tip of the retrieval bag 20. The tensile forces applied by the surgeon during withdrawal of the introducer with a closed retrieval bag 20 will typically be lower than tensile forces applied during withdrawal of a cinched retrieval bag 20 due to a difference in the size or volume of the specimens that are being extracted. But, in both instances, forces can be significant enough to create a potential for rupture of the retrieval bags 20. Tissue retrieval systems that are not redeployable and therefore designed for the withdrawal of specimens only after the retrieval bag has been cinched and detached from the introducer include retrieval bag configurations designed to withstand the associated tensile forces but are not designed to withstand the tensile forces that a redeployable retrieval bag can experience during withdrawal of a closed retrieval bag that is attached to the introducer.

In certain embodiments, it is desirable that the tissue retrieval systems described herein include a retrieval bag that is practical and simple to manufacture that is configured to withstand the tensile forces encountered during the different operational conditions encountered by a redeployable tissue retrieval bag. Thus, it is desirable that a redeployable tissue retrieval bag be configured to withstand tensile forces induced by a surgeon during withdrawal of small specimens such as lymph nodes with the retrieval bag in the closed configuration and attached to the introducer and that is configured to withstand the tensile forces induced by the surgeon during the withdrawal of large specimens such as a gallbladder or kidney with the retrieval bag in the cinched configuration and detached from the introducer.

In various embodiments a redeployable tissue retrieval bag 20 can be configured to withstand the typical tensile forces induced by surgeons during the serial withdrawal of multiple small specimens where the retrieval bag 20 is in the closed configuration and is attached to the actuator 7 and that is also configured to withstand the typical tensile forces induced by surgeons during the withdrawal of large specimens where the retrieval bag 20 is in the cinched configuration and is detached from the actuator 7. In certain embodiments, a redeployable tissue retrieval bag 20 for use with tissue retrieval systems described herein can include a strengthened proximal upper portion where the bead 50 is attached to enable the reversible partial retraction of the retrieval bag 20 into the introducer 3 tube and closure of the retrieval bag 20 to contain a small specimen for subsequent withdrawal from a patient.

With reference to FIGS. 8-9, an embodiment of redeployable tissue retrieval bag 20 is illustrated. FIG. 8 illustrates the tissue retrieval bag 20 with a bead 50 coupled thereto by an annular clamp 53. FIG. 9 illustrates tissue retrieval bag 20 with the bead 50 removed. In the illustrated embodiment, the tissue retrieval bag 20 comprises an open end 120 and a closed end 122 opposite the open end. A cuff 124 extends along the open end 120 and extends longitudinally from a proximal end to a distal end with respect to the central longitudinal axis L of the introducer when the retrieval bag 20 is in a stowed position or a deployed position. The cuff 124 is sized and configured to receive the support arms and the cord loop 42 therein. A depth axis D extends orthogonal to the central longitudinal axis L between the open end 120 and the closed end 122.

With continued reference to FIGS. 8-9, the illustrated embodiment of tissue retrieval bag further comprises a distal edge 126 extending from the distal end of the cuff 124 at the open end 120 to the closed end 122. A proximal edge 128 extends from the proximal end of the cuff 124 to the closed end 122. In the illustrated embodiment, the retrieval bag 20 is formed from a sheet of material having a fold in the sheet defining the distal edge 126 and a welded seal defining the closed end 122 and the proximal edge 128. In other embodiments, a proximal edge can be formed at a folded sheet and a distal edge can be welded. In still other embodiments, a tissue retrieval bag can be formed of multiple sheets that are adhered or welded to one another.

With continued reference to FIGS. 8-9, the illustrated embodiment of tissue retrieval bag 20 further comprises an attachment tab or proximal upper portion 130. In the illustrated embodiment, the proximal upper portion 130 is positioned at the open end 120 and extends transversely to the central longitudinal axis L in a direction proximal with respect to the central longitudinal axis and away from the closed end 122 with respect to the depth axis D. In certain embodiments, the proximal upper portion is aligned with or extends along an axis that is colinear to an axis defined by the proximal edge 128. As illustrated in FIG. 8, the bead 50 is coupled to the proximal upper portion 130.

With continued reference to FIGS. 8-9, the proximal upper portion 130 comprises a lower edge 132 and an upper edge 134 opposite the lower edge. The lower edge 132 can be aligned with the proximal edge 128 of the tissue retrieval bag 20. In the illustrated embodiment the lower edge 132 is colinear with the proximal edge 128. For example, in certain embodiments, the lower edge 132 can be formed by a continuation of the welded seam of the proximal edge 128 over the proximal upper portion 130. Advantageously, such aligned or colinear arrangements of the proximal upper portion 130, to which the bead 50 is attached, can significantly reduce stress concentrations which may otherwise result adjacent the bead 50 in a redeployable tissue retrieval bag.

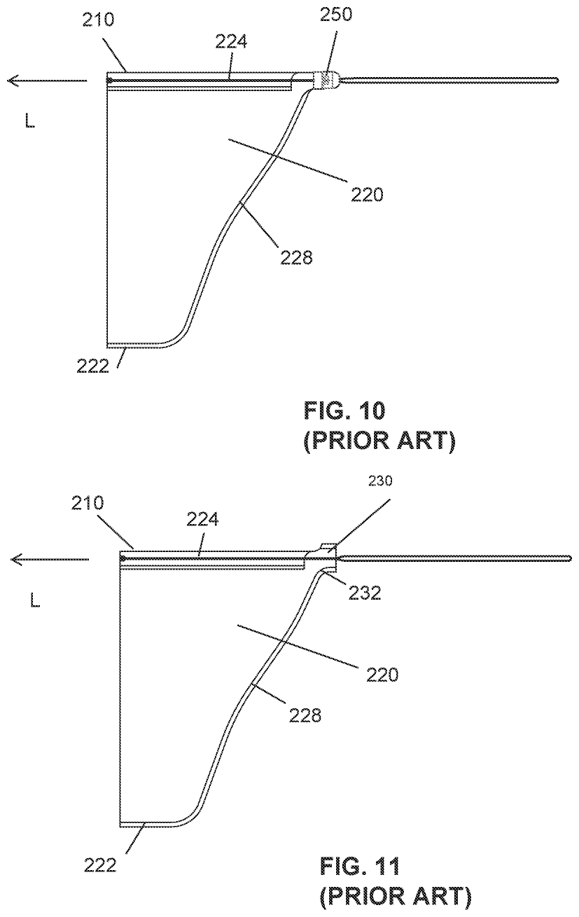

With reference to FIGS. 10-11, an exemplary embodiment of prior art tissue retrieval bag 220 is illustrated. FIG. 10 illustrates the tissue retrieval bag 220 with a bead 250 coupled thereto. FIG. 11 illustrates tissue retrieval bag 220 with the bead 250 removed. In the illustrated embodiment, the tissue retrieval bag 220 comprises an open end 210 and a closed end 222 opposite the open end. A cuff 224 extends along the open end 210 and extends longitudinally from a proximal end to a distal end with respect to the central longitudinal axis L of the introducer when the retrieval bag 220 is in a stowed position or a deployed position. The cuff 224 is sized and configured to receive the support arms and the cord loop therein.

With continued reference to FIGS. 10-11, in the exemplary prior art tissue retrieval bag 220, the bead 250 is coupled to a proximal portion 230 of the retrieval bag 220 that extends from the proximal end of the cuff 224 longitudinally proximally with respect to the central longitudinal axis L. Thus, the proximal portion 230 extends longitudinally, parallel to the cuff 224 and the open end of the retrieval bag 220. The illustrated retrieval bag 220 comprises a proximal edge 228 extending from a proximal end of the open end 210 transversely to the central longitudinal axis L to the closed end 222. In the illustrated prior art embodiment, the proximal portion 230 is blended into the proximal edge 228 of the retrieval bag via a radius 232. In the exemplary embodiment of tissue retrieval bag 220 for a single deployment, the proximal portion 230 extends longitudinally with respect to the central longitudinal axis L to enable installation of the support arms and to align with the support arms and the longitudinal axis of the introducer tube. The radius 232 blending the longitudinal proximal portion 230 with the proximal edge 228 of the retrieval bag represents a stress raiser that results in a stress concentration at the location of the radius. If this exemplary embodiment of prior art retrieval bag 220 is utilized in a redeployable tissue retrieval system and is then partially retracted into the introducer tube to close and contain the open end 210, the stress concentration at the radius 232 can result in the retrieval bag rupturing or tearing at a relatively low force when the surgeon is trying to extract the contained specimen.

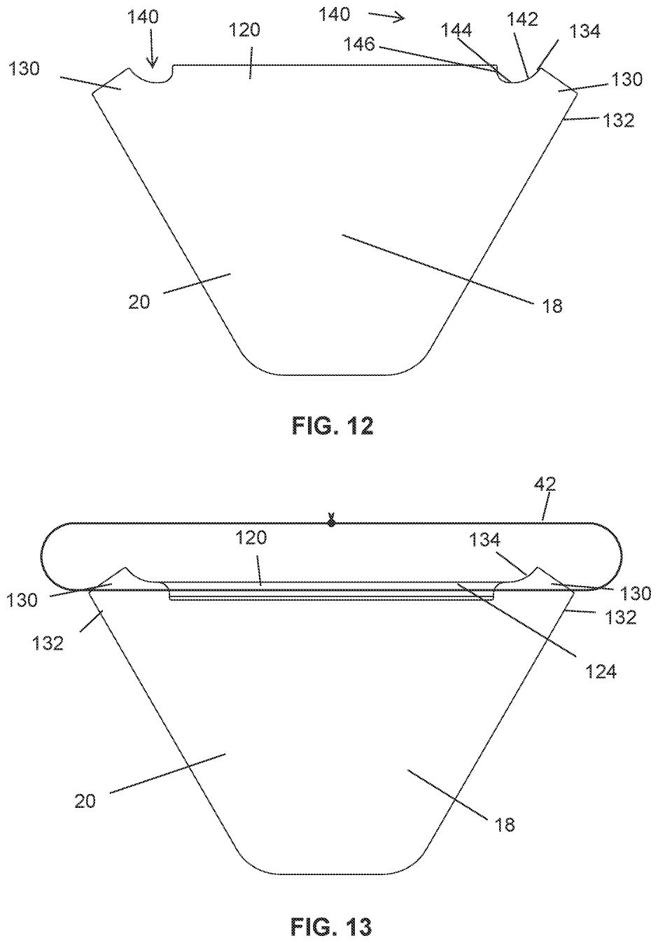

With reference to FIGS. 12-13, schematic views of a sheet of material 18 are illustrated as initially cut, such as by a die cut pattern (FIG. 12) and partially formed (FIG. 13) to a retrieval bag 20 such as that of FIGS. 8-9. To form the retrieval bags 20 described herein for use with redeployable tissue retrieval systems, the sheet of material 18 can be cut to define an open end 120 having a cuff 124.

With continued reference to FIGS. 12-13, in the illustrated embodiment, the retrieval bag 20 comprises a proximal upper portion 130 that is configured to provide an attachment tab for the bead that provides a relatively high tensile strength during deployment cycles. For example, the proximal upper portion 130 comprises a lower edge 132 that is substantially colinear with or aligned with a proximal edge of the tissue retrieval bag. Accordingly, the lower edge 132 of the proximal upper portion 130 or attachment tab is configured to provide a relatively high tensile strength with substantially no stress concentrations. Since the aligned or colinear arrangement of the lower edge 132 of the upper proximal portion 130 is able to withstand relatively high tensile forces, in certain embodiments, it is desirable that upper edge 134 of the upper proximal portion 130 likewise be configured to minimize stress concentrations therein to reduce the tendency for the retrieval bag 20 to rupture therein at a radiused transition area from the upper proximal portion 130 to the cuff 124. In certain embodiments, the proximal upper portion 130 further comprises an upper edge 134 that comprises a relatively large radius on each side extending from just beyond the bead section to minimize a stress concentration in this area.

With continued reference to FIGS. 12-13, in certain embodiments, the proximal upper portion 130 comprises an upper edge 134 having a radiused profile 140 to minimize stress concentrations during tensile loading cycles. For example, in the illustrated embodiment, the radiused profile 140 comprises a first radiused portion 142 adjacent the bead 50, a second radiused portion 144, and a transition portion 146 adjacent the cuff. The first radiused portion 142 is positioned proximally of the second radiused portion 144, and the second radiused portion 144 is positioned proximally of the transition portion 146 with the tissue retrieval bag in a formed state. (FIG. 8-9). The first radiused portion 142 can have a first radius. The second radiused portion can have a second radius. In certain embodiments, the first radius is relatively large compared to the second radius. In certain embodiments, the first radius is desirably at least about 0.5 inches, and more desirably about 0.75 inches. In certain embodiments, the second radius is approximately 0.25 inches. Desirably, the first radiused portion with a relatively large radius provides a high tensile strength by reducing stress concentrations at the upper edge of the attachment tab or proximal upper portion 130 for a redeployable tissue retrieval bag 20. As the relatively large radii of the first radiused portion 142 approach the cuff 124 of the retrieval bag 20, the large radii tangentially transition into relatively smaller radii of the second radiused portion 144 that then tangentially transition at the transition portion 146 into vertical edges that form the cuff openings on each side of the retrieval bag 20. In this radiused profile, it is desirable that the first radiused portion extends beyond where the fold starts that forms the cuff. Desirably, this configuration results in the smaller radius becoming part of the cuff, which is a stronger section of the bag due to the cuff being formed of two layers of material. As illustrated, on the pouch pattern to cut a sheet of material to form a redeployable tissue retrieval bag, the radiused profile 140 of the upper edge 134 of the larger radii transition into the smaller radii and then into a vertical direction to form a half teardrop shape similar to the shape of a teardrop trailer.

With continued reference to FIGS. 12-13, the radiused profile 140 of the upper edge 134 in the illustrated embodiment, including transitioning of the relatively large radii to the relatively smaller radii and then to the vertical edge advantageously enables the cuff openings to be closer to the bead as compared to a configuration with only the large radii. Desirably, with the cuff openings closer to the bead, the cord loop and the supports can be more substantially contained within the cuff.

As illustrated in FIG. 13, to form a retrieval bag, a portion of the cut sheet of material forming the cuff 124 is folded over the pre-tied cord loop and then welded in place to trap the cord loop 42 within the cuff 124. The sheet of material is then folded in half lengthwise to form the distal edge of the tissue retrieval bag, welded to form the closed end and the proximal edge, and cut to form the final shape of the retrieval bag. During this second welding step, a lower edge of the proximal upper portion 130 of the retrieval bag 20 can also be welded. Accordingly, a lower edge of the proximal upper portion 130 can comprise a welded seam that is continuous with a welded seam of the proximal edge such that the lower edge of the proximal upper portion is colinear with the proximal edge of the tissue retrieval bag. This forming process for the retrieval bag is simple and cost effective and results in a retrieval bag with a high tensile strength. Additional material reinforcements, stitching, or other secondary operations can add cost and complexity for the assembly process and may not be necessary to further improve the tensile loading capabilities of a redeployable tissue retrieval bag. The bead 50 and annular clamp 53 can be attached to the retrieval bag 20 using adhesive, heat sealing, ultrasonic welding, or similar processes. (FIG. 8). In other embodiments, the bead can be formed in two halves to enable the mechanical attachment of the bead to the retrieval bag.

Empirical test data comparing a prior art retrieval bag 220 configuration, such as that illustrated in FIGS. 10-11 with a redeployable tissue retrieval bag 20 of FIGS. 8-9, with both retrieval bags being formed from the same film material, demonstrates the improved strength of the present invention. During tensile testing of the exemplary prior art retrieval bag 220, it was observed that the retrieval bag 220 tore at the lower radius 232 that blends the longitudinal upper proximal portion with the downward extending portion at a relatively low average force. During tensile testing of the redeployable tissue retrieval bag 20, it was observed that the retrieval bag 20 eventually tore at the radiused profile 140 of the upper edge 134 of the upper proximal portion 130 at a relatively high average force, representing more than a fourfold increase in the average tensile strength of the present invention as compared to the prior art.

Thus, advantageously, the redeployable tissue retrieval bag 20 described herein significantly reduces stress concentrations adjacent a proximal portion of the tissue retrieval bag by, among other aspects, eliminating a radius 232 blending a proximal edge with a proximal portion to which a bead is attached and the corresponding stress concentration. The tissue retrieval bag 20 includes an angled upper proximal portion 130 that extends transversely at the same angle relative to the central longitudinal axis as the proximal edge of the tissue retrieval bag 20 such that there is no radius and corresponding stress concentration in this portion of the retrieval bag. When the support arms are inserted through the bead 50 and into the cuff 124 during assembly of the device, the upper proximal portion 130 of the tissue retrieval bag 20 with the attached bead 50 rotates clockwise and folds over due to the flexibility of the retrieval bag film material such that it is aligned with the cuff. The installed support arms maintain the upper proximal portion 130 of the retrieval bag 20 and the attached bead 50 in a longitudinally aligned state with the introducer tube. (FIG. 1).

In various embodiments, the retrieval bag 20 can be formed from a pouch pattern which is die-cut from a sheet of film material. The film material can be comprised of a variety of materials including mylar, polyester, polyethylene, polyimide, polyurethane, ripstop nylon, ripstop polyester, or other similar materials. The film material could also be comprised of a variety of laminated or coated materials such as polyurethane coated or laminated ripstop polyester, polyurethane coated or laminated spandex, polyurethane coated or laminated ripstop nylon, polyurethane coated or laminated taffeta, silicone coated ripstop polyester, silicone coated ripstop nylon, or other similar materials. Preferably, the pattern is formed from a material that can be welded or heat sealed to itself. A ripstop fabric with a polymer material coating or laminate can typically be welded or heat sealed to itself.

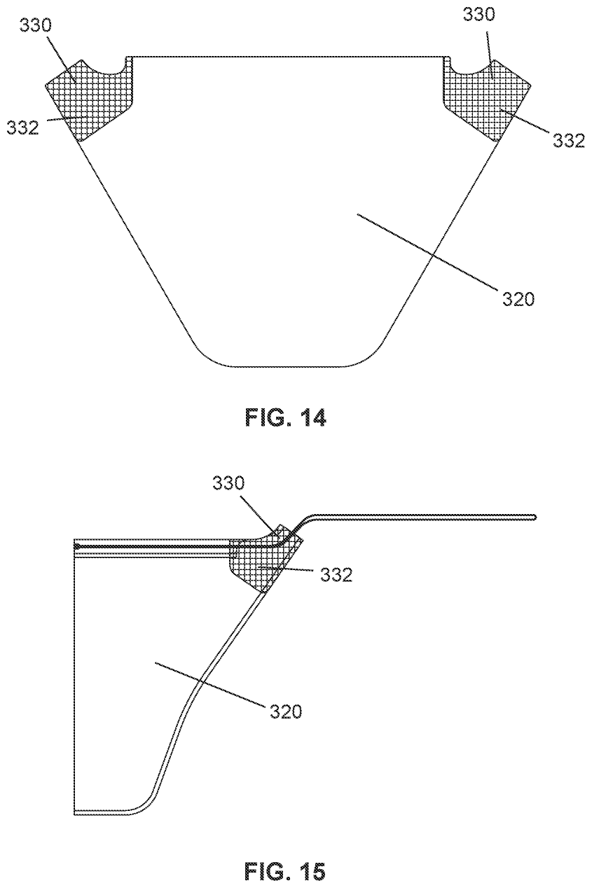

With reference to FIGS. 14-15, another embodiment of redeployable tissue retrieval bag 320 is illustrated. FIG. 14 illustrates a sheet of material that can be formed into a redeployable tissue retrieval bag 320. FIG. 15 illustrates a partially formed tissue retrieval bag 320 before a bead has been coupled thereto. As illustrated, the tissue retrieval bag includes additional layers of material reinforcements 332 in the upper proximal portion 330 of the retrieval bag to provide tensile increased strength when tensioned in the closed configuration. As illustrated, the tissue retrieval bag 320 includes material reinforcements 332 positioned over an upper proximal portion 330 that is substantially similar to the configuration of upper proximal portion 130 of the redeployable tissue retrieval bag 20 of FIGS. 12-13. In other embodiments, material reinforcements can be added to a proximal portion of a tissue retrieval bag similar to the exemplary prior art tissue retrieval bag illustrated in FIGS. 10-11. The additional layers of reinforcement material 332 can desirably be formed from a single sheet of weldable material such as polyurethane or, in other embodiments, the additional layers can comprise a three-layer laminate which can include two outer layers of a weldable material such as polyurethane and an inner layer such as ripstop nylon. In the illustrated embodiment, the reinforcement material 332 is welded to each upper proximal portion 330 of the sheet of material forming the tissue retrieval bag 320 and then welded together along the seams when the sheet of material is folded over and welded to form the retrieval bag 320.

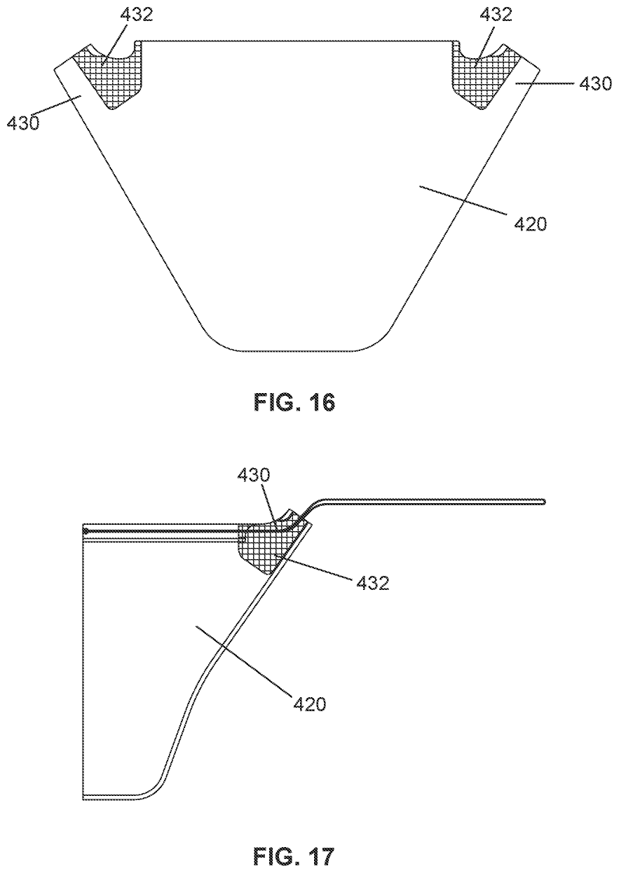

With reference to FIGS. 16-17, another embodiment of redeployable tissue retrieval bag 420 is illustrated. FIG. 16 illustrates a sheet of material that can be formed into a redeployable tissue retrieval bag 420. FIG. 17 illustrates a partially formed tissue retrieval bag 420 before a bead has been coupled thereto. In the illustrated embodiments, additional reinforcement material 432 can be formed from the same material as the retrieval bag 420. For a retrieval bag formed from a ripstop nylon and polyurethane laminate material, since the ripstop nylon material is laminated to a polyurethane film, the material reinforcements can only be welded to the polyurethane side of the laminate if the peel strengths of the seams in the reinforced area are to be optimized. The reinforcement material 432, if made from the same material as the retrieval bag, can only be welded on one side as well. In certain embodiments formed of a nylon laminate material, if the reinforcement material 432 is welded to each upper proximal portion 430 of the sheet of material cut from a pouch pattern, when the sheet of material is then folded over for welding, it is not possible to weld the pouch pattern in the reinforced areas since the ripstop nylon side of the laminate reinforcements cannot be welded together. In the illustrated embodiment, to overcome this limitation, the reinforcement material 432 can advantageously be cut to size and welded to the sheet of material such that they do not cover the seam areas of the sheet of material along the proximal edge and lower edge of the upper proximal portion that are subsequently welded together after the pouch pattern is folded over allowing the seam areas to be welded together to form the retrieval bag. In other embodiments, the reinforcement material 432 can be welded to the formed retrieval bag 420 after welding. In these embodiments, the formed retrieval bag 420 would be turned inside out and the reinforcement material 432 would be welded to the inside upper proximal portion 430 of the retrieval bag between the seams to provide the reinforced areas.

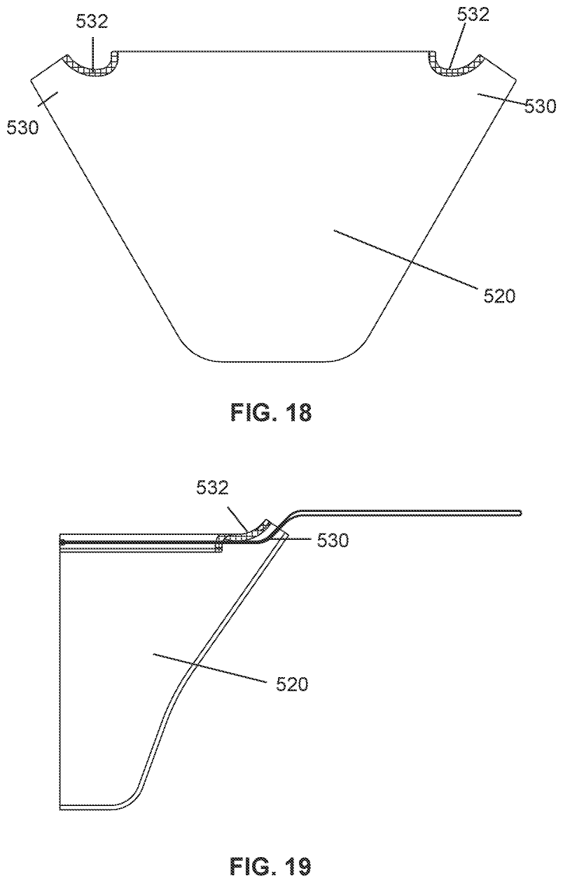

With reference to FIGS. 18-19, another embodiment of redeployable tissue retrieval bag 520 is illustrated. FIG. 18 illustrates a sheet of material that can be formed into a redeployable tissue retrieval bag 520. FIG. 19 illustrates a partially formed tissue retrieval bag 520 before a bead has been coupled thereto. In the illustrated embodiments, additional reinforcement material 532 can be added along some or all of the upper edge of the upper proximal portion 530. For example, in the illustrated embodiment, the additional reinforcement material 532 is added along the radiused profile of the upper edge on both sides to reduce the stresses and further increase the strength of the retrieval bag 520 in this area. In other embodiments, the reinforcement material 532 can be added to only some of the first radiused portion, the second radiused portion, and the transition portion of the radiused portion.

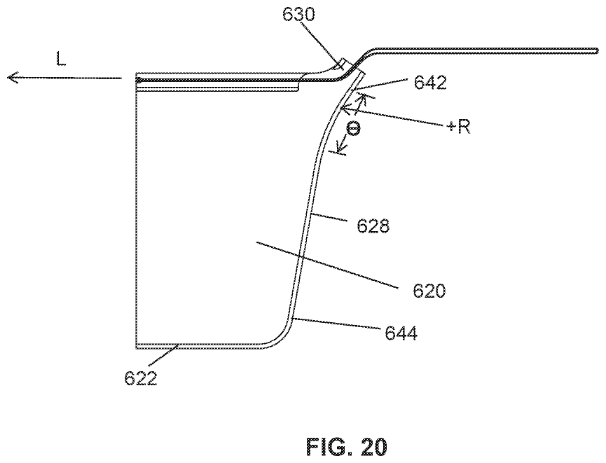

Certain embodiments of redeployable tissue retrieval bags have been described and illustrated herein as having a proximal edge that is transverse to the central longitudinal axis and generally linear such that a proximal upper portion can extend transverse to the central longitudinal axis and colinear with the proximal edge. With reference to FIG. 20, in other embodiments, a redeployable tissue retrieval bag 620 can comprise a proximal upper portion 630 extending transversely to the central longitudinal axis L and a proximal edge 628 having a relatively large radiused profile having a relatively large radius R and transcribing an angular arc θ. For example, in these embodiments, an upper portion 642 of the proximal edge can be substantially colinear with the upper proximal portion 630 extending transverse to the central longitudinal axis, while a lower portion 644 of the proximal edge can be substantially perpendicular to the closed end 622 of the tissue retrieval bag, with the proximal edge 628 having a radiused profile between the upper portion and the lower portion. In certain embodiments, the proximal edge 628 has a radiused profile with a radius R of approximately 4 inches over an angular arc θ of approximately 155 degrees. In other embodiments, the radius R or the radiused profile can be greater than 4 inches. In certain other embodiments, the angular arc can be greater than 155 degrees such as, for example, up to 180 degrees. Desirably, such an arrangement can facilitate a relatively large retrieval bag volume while maintaining tensile strength at the upper proximal portion and along the proximal edge for redeployable use of the tissue retrieval bag.

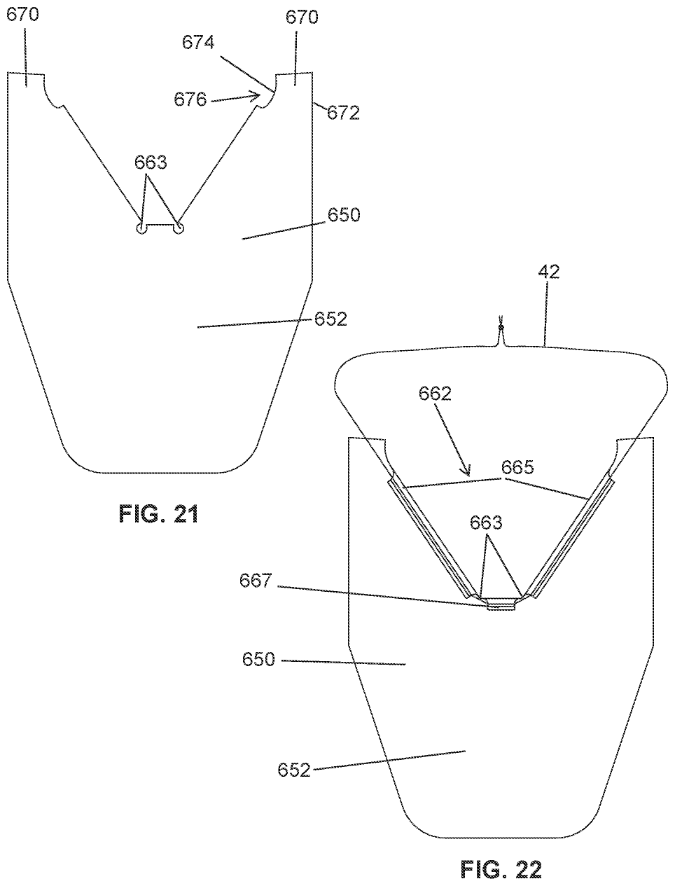

With reference to FIGS. 21-40, certain embodiments of tissue retrieval bag assemblies in various stages of manufacture and assembly are illustrated. These embodiments of tissue retrieval bags can be used in conjunction with a tissue retrieval system of FIGS. 1-7. The tissue retrieval bag assemblies of FIGS. 21-40 are configured to be repeatably redeployable with a relatively low stress concentration configuration. Additionally, the tissue retrieval bag assemblies of FIGS. 21-40 further comprise an elongate profile. These tissue retrieval bag assemblies have an open end defined by a cuff and a closed end opposite the open end. The cuff has a proximal end and a distal end defining a longitudinal axis therebetween that extends along the longitudinal axis of the tubular introducer with the tissue retrieval bag in a stowed configuration. The elongate profile of the tissue retrieval bags is defined by a portion of the tissue retrieval bag extending distal to the distal end of the cuff. In the illustrated embodiments, the tissue retrieval bags extend distally from the distal end of the cuff along a bag axis, B transverse to the longitudinal axis of the cuff. Advantageously, a tissue retrieval bag having such a longitudinal profile can have a relatively low retraction force to retract the bag to a stowed configuration as compared with a tissue retrieval bag of equivalent volume without an elongate profile. Moreover, in certain embodiments, a tissue retrieval bag having an elongate profile can be rolled to an initial stowed configuration having a relatively small diameter as compared with a tissue retrieval bag of equivalent volume without an elongate profile. Accordingly, these embodiments of tissue retrieval system can be particularly well suited for use in relatively small diameter (for example, 5 mm laparoscopic system) tissue retrieval systems by allowing relatively large bag storage volumes and relatively small diameter stowed configurations. Alternatively, these embodiments of tissue retrieval system can be used in larger diameter tissue retrieval systems (for example, 10-15 mm laparoscopic systems) to provide a tissue retrieval bag having a relatively large bag storage volume as compared with a tissue retrieval system of equivalent diameter without an elongate profile.

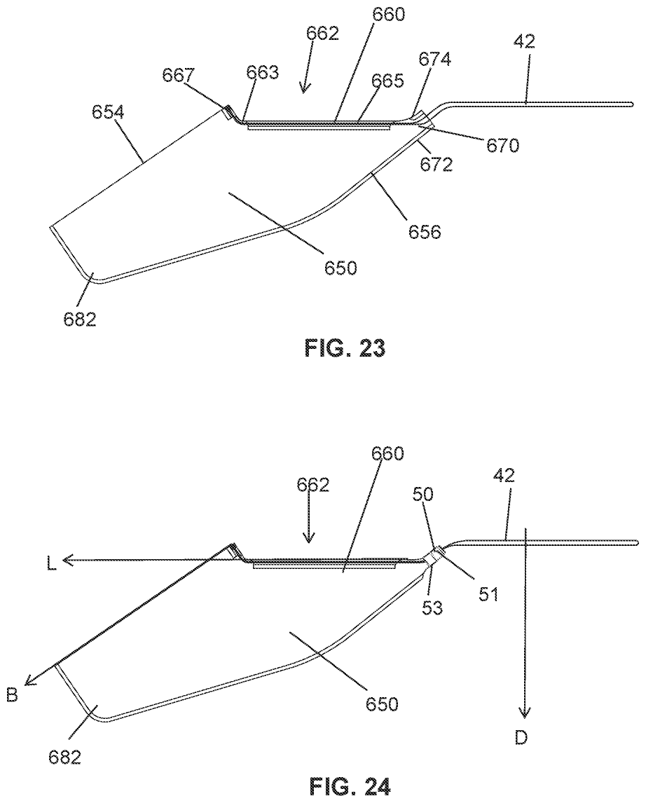

With reference to FIGS. 21-22, schematic views of a sheet of material 652 are illustrated as initially cut, such as by a die cut pattern (FIG. 21) and partially formed (FIG. 22) to a retrieval bag 650 such as further illustrated in FIGS. 23-24. To form the retrieval bags 650 described herein for use with redeployable tissue retrieval systems, the sheet of material 652 can be cut to define an open end 660 having a cuff 662 and a tab or proximal upper portion 670.

With continued reference to FIGS. 21-22, in the illustrated embodiment, the retrieval bag 650 comprises a proximal upper portion 670 that is configured to provide an attachment tab for the bead that provides a relatively high tensile strength during deployment cycles. For example, the proximal upper portion 670 comprises a lower edge 672 that is substantially colinear with or aligned with a proximal edge of the tissue retrieval bag. Accordingly, the lower edge 672 of the proximal upper portion 670 or attachment tab is configured to provide a relatively high tensile strength with substantially no stress concentrations. Since the aligned or colinear arrangement of the lower edge 672 of the upper proximal portion 670 is able to withstand relatively high tensile forces, in certain embodiments, it is desirable that upper edge 674 of the upper proximal portion 670 likewise be configured to minimize stress concentrations therein to reduce the tendency for the retrieval bag 650 to rupture therein at a radiused transition area from the upper proximal portion 670 to the cuff 662. In certain embodiments, the proximal upper portion 670 further comprises an upper edge 674 that comprises a relatively large radius on each side extending from just beyond the bead section to minimize a stress concentration in this area.

With continued reference to FIGS. 21-22, in certain embodiments, the proximal upper portion 670 comprises an upper edge 674 having a radiused profile 676 to minimize stress concentrations during tensile loading cycles. In certain embodiments, the radiused profile 676 can be configured as discussed with respect to the tissue retrieval bags of FIGS. 8-9. In other embodiments, the upper portion 670 can comprise a proximal edge having a radiused profile transcribing an angular arc as discussed with respect to the tissue retrieval bags of FIG. 20.

As illustrated in FIG. 22, to form a retrieval bag, a portion of the cut sheet of material forming the cuff 662 is folded over the pre-tied cord loop and then welded in place to trap the cord loop 42 within the cuff 662. In the illustrated embodiment, the retrieval bag has an elongate profile with a closed end positioned distal, relative to a longitudinal axis defined by the cuff 662, of the open end. In the illustrated embodiment, the cuff 662 is formed as a non-continuous cuff comprising a pair of side segments 665 extending distally along the cuff axis from the proximal end of the cuff 662 and an end segment 667 at the distal end of the cuff. Thus, when the material forming the cuff 662 is formed, each of the side segments 665 and the end segment 667 of the cuff 662 are folded over the cord loop 42 and welded in place.

As illustrated in FIG. 21, the die cut pattern comprises two cutouts or recesses 663 therein. Each recess 663 is positioned at a distal end of a side segment 665 of the cuff such that each recess 663 defines an end of the end segment 667 of the cuff. Each recess 663 can comprise a radiused edge such that stress concentrations at the recess are reduced.

The sheet of material is then folded in half (FIG. 23) lengthwise to form the distal edge of the tissue retrieval bag, welded to form the closed end and the proximal edge, and trimmed to form the final shape of the retrieval bag. The folded distal edge extends along a bag axis, B that extends transversely to the longitudinal axis defined by the cuff 662 such that a portion of the tissue retrieval bag is positioned distal to the opening defined by the cuff 662. During this second welding step, a lower edge of the proximal upper portion 670 of the retrieval bag 20 can also be welded. Accordingly, a lower edge of the proximal upper portion 670 can comprise a welded seam that is continuous with a welded seam of the proximal edge such that the lower edge of the proximal upper portion is colinear with the proximal edge of the tissue retrieval bag. This forming process for the retrieval bag is simple and cost effective and results in a retrieval bag with a high tensile strength. Additional material reinforcements, stitching, or other secondary operations can add cost and complexity for the assembly process and may not be necessary to further improve the tensile loading capabilities of a redeployable tissue retrieval bag. The bead 50 and annular clamp 53 can be attached to the retrieval bag 650 using adhesive, heat sealing, ultrasonic welding, or similar processes. (FIG. 24). In other embodiments, the bead can be formed in two halves to enable the mechanical attachment of the bead to the retrieval bag.

With reference to FIG. 24, in certain embodiments, the bead 50 can comprise a two-piece assembly comprising a bead body 51 and an annular clamp 53 positioned around a portion of the bead body. A portion of the tissue retrieval bag can be positioned between the bead body 51 and the clamp 53 to couple the tissue retrieval bag to the bead 50. Various joining techniques such as gluing with chemical adhesives, ultrasonic welding, heat sealing, or other chemical, thermal, or mechanical joining processes can further secure the retrieval bag to the bead body and/or clamp. In other embodiments, the bead can be a single component without an encircling clamp, and the single component bead can be coupled to the retrieval bag with various chemical, thermal, or mechanical joining processes.

With reference to FIGS. 23-24, an embodiment of redeployable tissue retrieval bag 650 is illustrated. FIG. 23 illustrates tissue retrieval bag 650 with the bead 50 removed. FIG. 24 illustrates the tissue retrieval bag 650 with a bead 50 coupled thereto by an annular clamp 53. In the illustrated embodiment, the tissue retrieval bag 650 comprises an open end 660 and a closed end 682 opposite the open end. A cuff 662 extends along the open end 660 and extends longitudinally from a proximal end to a distal end defining a cuff axis which extends along the central longitudinal axis L of the introducer when the retrieval bag 20 is in a stowed position. The cuff 662 is sized and configured to receive the support arms and the cord loop 42 therein. A depth axis D extends orthogonal to the central longitudinal axis L between the open end 660 and the closed end 682. A distal edge of the tissue retrieval bag 650 extends along a bag axis, B transverse to the longitudinal axis L such that a portion of the tissue retrieval bag 650 is distal of the open end 660 and the cuff 662.

With continued reference to FIGS. 23-24, the illustrated embodiment of tissue retrieval bag 650 further comprises a distal edge 654 extending from the distal end of the cuff 662 at the open end 660 to the closed end 682. A proximal edge 656 extends from the proximal end of the cuff 662 to the closed end 682. In the illustrated embodiment, the retrieval bag 650 is formed from a sheet of material having a fold in the sheet defining the distal edge 654 and a welded seal defining the closed end 682 and the proximal edge 656. In other embodiments, a proximal edge can be formed at a folded sheet and a distal edge can be welded. In still other embodiments, a tissue retrieval bag can be formed of multiple sheets that are adhered or welded to one another.

With continued reference to FIGS. 23-24, the illustrated embodiment of tissue retrieval bag 650 further comprises an attachment tab or proximal upper portion 670. In the illustrated embodiment, the proximal upper portion 670 is positioned at the open end 660 and extends transversely to the central longitudinal axis L in a direction proximal with respect to the central longitudinal axis and away from the closed end 682 with respect to the depth axis D. In certain embodiments, the proximal upper portion is aligned with or extends along an axis that is colinear to an axis defined by the proximal edge 656. As illustrated in FIG. 24, the bead 50 is coupled to the proximal upper portion 670.

With continued reference to FIGS. 23-24, the proximal upper portion 670 comprises a lower edge 672 and an upper edge 674 opposite the lower edge. The lower edge 672 can be aligned with the proximal edge 656 of the tissue retrieval bag 650. In the illustrated embodiment the lower edge 672 is colinear with the proximal edge 656. For example, in certain embodiments, the lower edge 672 can be formed by a continuation of the welded seam of the proximal edge 656 over the proximal upper portion 670. Advantageously, such aligned or colinear arrangements of the proximal upper portion 670, to which the bead 50 is attached, can significantly reduce stress concentrations which may otherwise result adjacent the bead 50 in a redeployable tissue retrieval bag.

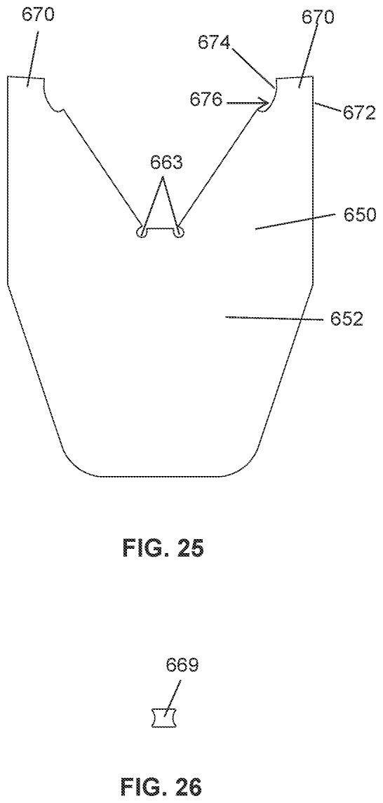

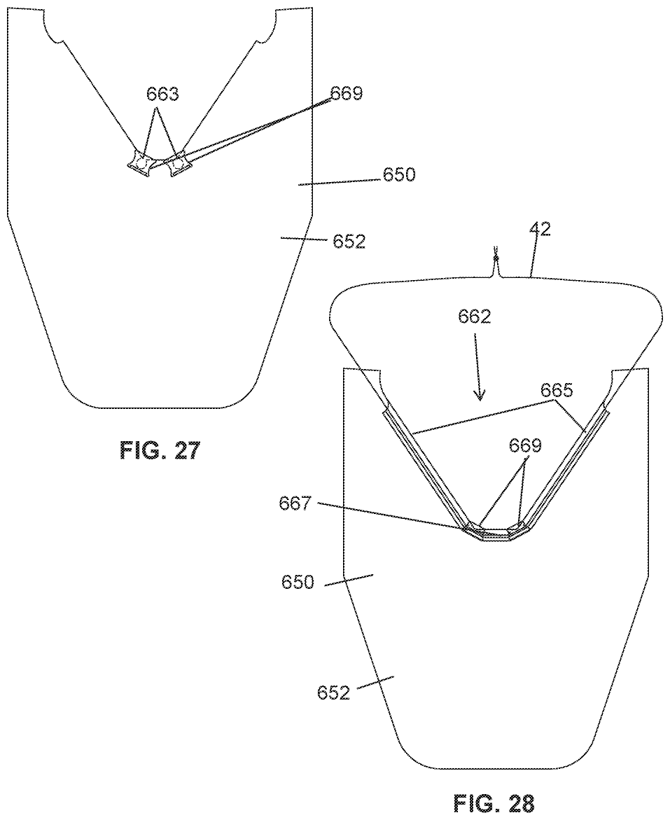

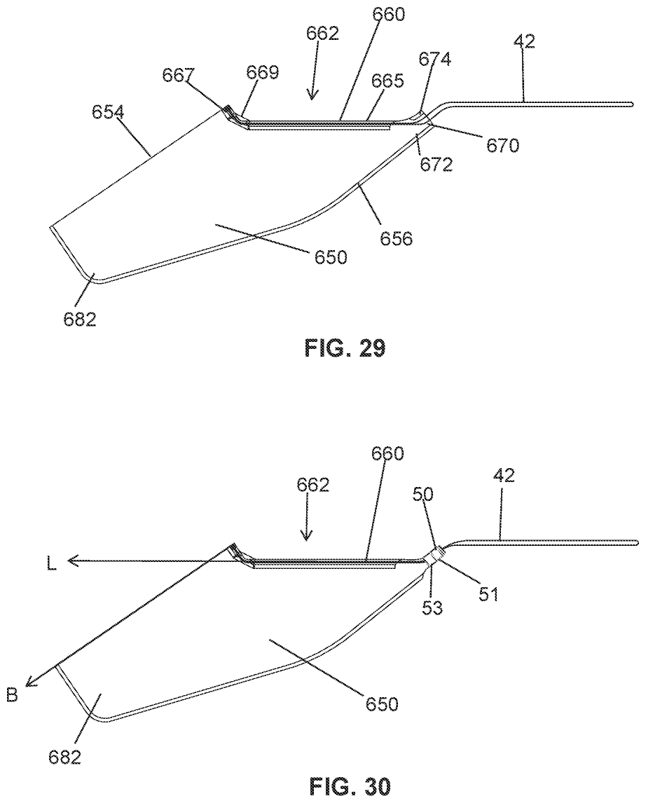

With reference to FIGS. 25-30 certain embodiments of the tissue retrieval bag 650 of FIGS. 21-24 can further comprise cuff reinforcement tabs 669 (FIG. 26) positioned at the cutouts 663 of the cuff 662 between the side segments 665 and end segment 667 of the cuff 662. With reference to FIGS. 25-28, schematic views of a sheet of material 652 are illustrated as initially cut, such as by a die cut pattern (FIG. 25) and partially formed (FIGS. 27-28) to a retrieval bag 650 such as further illustrated in FIGS. 29-30. FIG. 26 illustrates a schematic view of a cuff tab 669, which in the illustrated embodiments, is joined to the sheet of material 652 adjacent the recesses 663 (FIG. 27) and folded to define the cuff 662 (FIG. 28). To form the retrieval bags 650 described herein for use with redeployable tissue retrieval systems, the sheet of material 652 can be cut to define an open end 660 having a cuff 662 and a proximal upper portion 670.

With reference to FIG. 26, the cuff tab 669 is illustrated as a sheet of material cut or formed in a generally hourglass shape having a relatively narrowed mid-section and relatively wider end sections. In certain embodiments the cuff tab 669 can be formed of the same material and material thickness as the sheet of material 652 forming the tissue retrieval bag 650. In other embodiments, the cuff tab 669 can be formed of a different material and/or a material having a different thickness than the sheet of material 652.

With reference to FIGS. 27-28, in the illustrated embodiment, cuff tabs 669 are positioned to cover the recesses 663 formed in the sheet of material 652 between the side segments 665 and the end segment 667 of the cuff 662. The cuff tabs 669 are joined to the sheet of material 652 such as by welding (FIG. 27) to form a cuff assembly comprising a pair of side segments 665, the end segment 667, and a pair of cuff tabs 669. The cuff assembly is then folded to retain a cord loop 42 and joined to the sheet of material 652. (FIG. 28). The sheet of material can then be folded and joined as described with respect to the tissue retrieval bag of FIGS. 23-24.

With reference to FIGS. 29-30, an embodiment of tissue retrieval bag 650 substantially as discussed with respect to FIGS. 23-24 is illustrated. The tissue bag 650 of FIGS. 29-30 further comprises cuff tabs 669 extending between the distal ends of the side segments 665 of the cuff 662 and the ends of the end segment 667 of the cuff 662. Advantageously, these cuff tabs 669 can reinforce the cuff 662 at the distal end thereof. The reinforcements increase the tensile strength of the tissue retrieval bag to allow more force to be applied during extraction of the cinched bag through the abdominal wall.

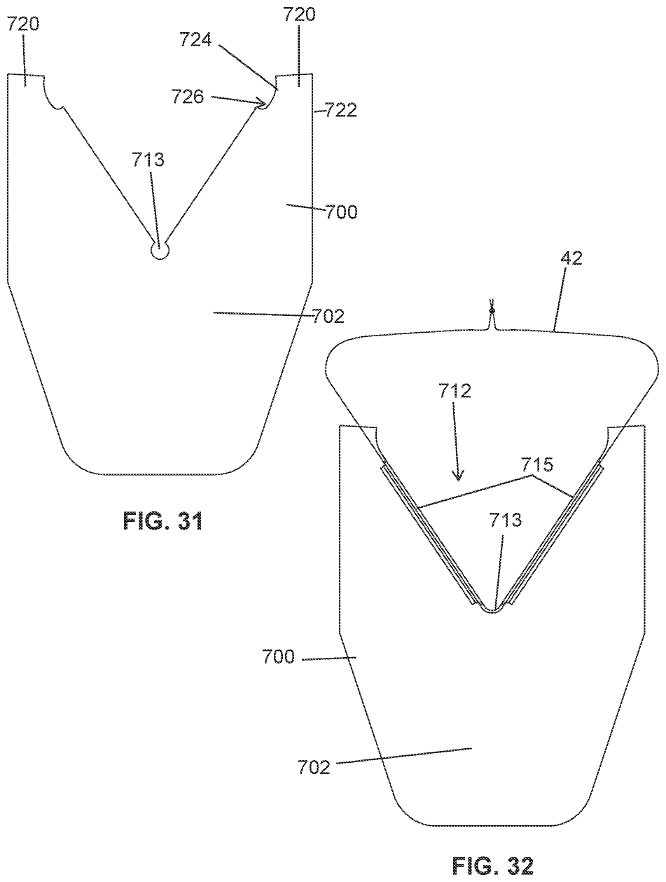

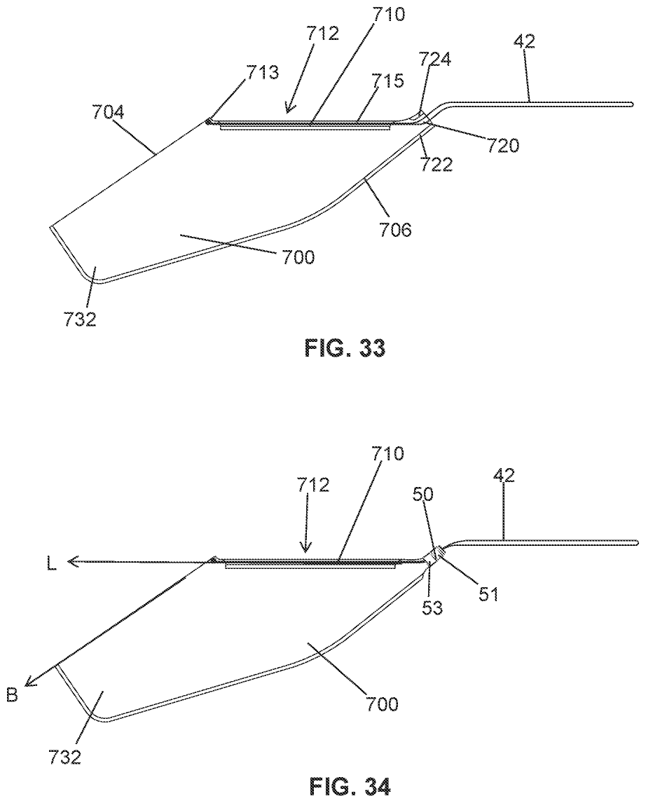

With reference to FIGS. 31-34, schematic views of a sheet of material 702 are illustrated as initially cut, such as by a die cut pattern (FIG. 31) and partially formed (FIG. 32) to a retrieval bag 700 such as further illustrated in FIGS. 33-34. To form the retrieval bags 700 described herein for use with redeployable tissue retrieval systems, the sheet of material 702 can be cut to define an open end 710 having a cuff 712 and a proximal upper portion 720.

With reference to FIGS. 31-34, the sheet of material 702 can be cut to form the tissue retrieval bag substantially as described with reference to the tissue retrieval bag 650 of FIGS. 21-24. Accordingly, in the illustrated embodiment, the sheet of material 702 and tissue retrieval bag 700 formed thereby can extend with an elongate profile along a bag axis, B between an open end 710 and a closed end 732. A bead 50 can be coupled to the tissue retrieval bag 700 at a tab configured to reduce stress concentrations and enhance redeployability of the tissue retrieval bag 700. As illustrated, the tab is defined by a proximal upper portion 720 having a lower edge 722 and an upper edge 724. Unlike the tissue retrieval bag 650 of FIGS. 21-24, tissue retrieval bag 700 comprises a cuff 712 having a single recess 713 or cutout that define a pair of side segments 715.

With continued reference to FIGS. 31-34, the recess 713 is cut into a portion of the sheet of material 702 that is folded to form the cuff 712 (FIG. 32). The recess 713 can create a noncontinuous cuff defined by two side segments 715. In the illustrated embodiment, the recess 713 is radiused to reduce a stress concentration. In other embodiments, other configurations of recess 713, such as with linear cuts, can be cut to form a noncontinuous cuff 712. The single recess 713 configuration can reduce the force to retract the tissue retrieval bag 700 into the introducer tube as compared to the double recess 663 configuration (FIGS. 21-24) as there is less material at the distal end of the cuff, which is typically the most difficult part of the tissue retrieval bag to retract into the introducer tube.

With reference to FIGS. 31-34, in one method for forming tissue retrieval bag 700, the side segments 715 of the cuff 712 can be folded to retain cord loop 42 and joined to the sheet of material 702. The sheet of material 702 can be folded to define a folded edge 704 extending along a bag axis, B transverse to an axis defined by the cuff. An opposite edge can be joined, such as by welding to define a sealed edge 706 of the tissue retrieval bag. A bead 50 can be joined to a tab at the proximal upper portion of the bag 720.

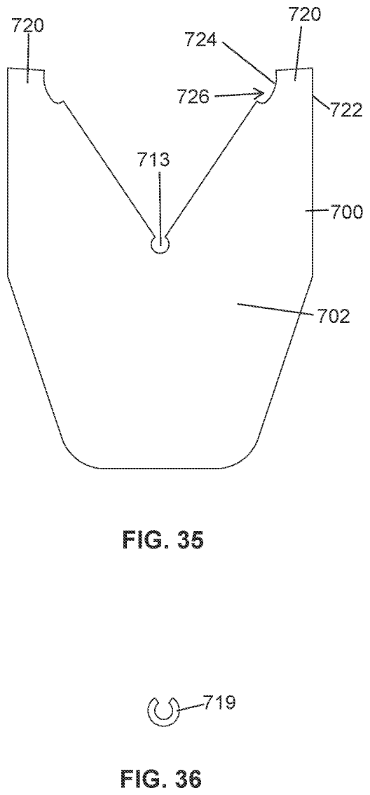

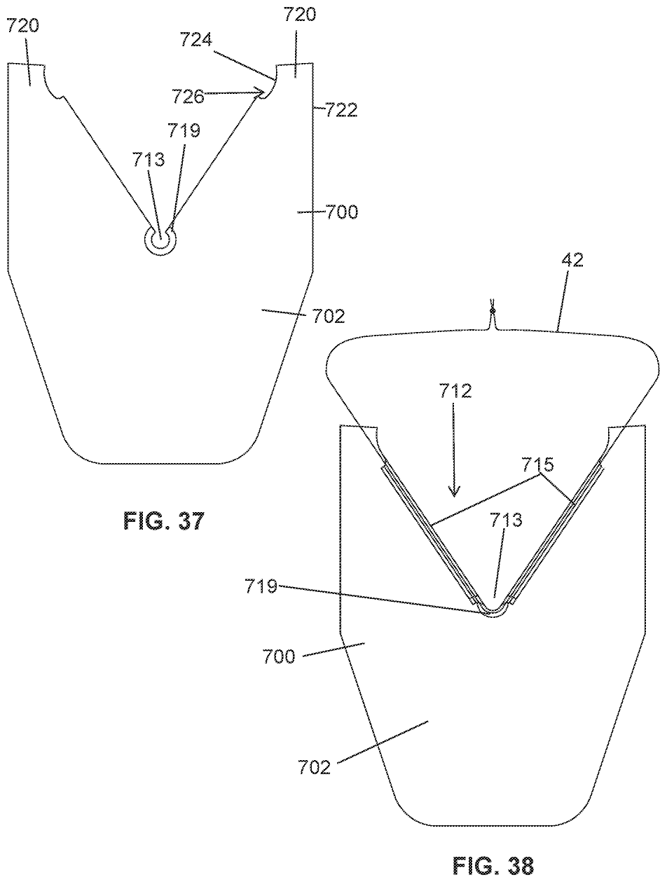

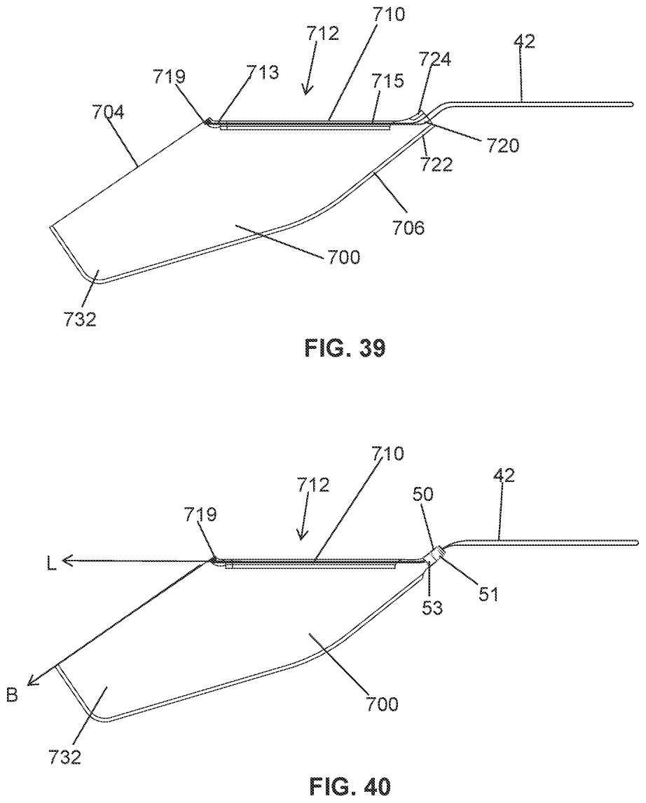

With reference to FIGS. 35-40, certain embodiments of the tissue retrieval bag 700 of FIGS. 31-34 can further comprise a cuff reinforcement such as a cuff ring 719 (FIG. 36) positioned at the recess 713 of the cuff 712 between the side segments 715 of the cuff 712. With reference to FIGS. 35-38, schematic views of a sheet of material 702 are illustrated as initially cut, such as by a die cut pattern (FIG. 35) and partially formed (FIGS. 37-38) to a retrieval bag 700 such as further illustrated in FIGS. 39-40. FIG. 36 illustrates a schematic view of a cuff ring 719, which in the illustrated embodiments, is joined to the sheet of material 702 adjacent the recess 713 (FIG. 37) and folded to define the cuff 712 (FIG. 38). To form the retrieval bags 700 described herein for use with redeployable tissue retrieval systems, the sheet of material 712 can be cut to define an open end 710 having a cuff 712 and a proximal upper portion 720.

With reference to FIG. 36, the cuff ring 719 is illustrated as a sheet of material cut or formed in a generally annular or ring shape with a recess or notch formed in a segment of the ring shape. In the illustrated embodiment, the cuff ring 719 has a radius generally corresponding to a radius of the recess 713 of the cuff 712 such that the cuff ring 719 can be positioned adjacent the recess 713. In certain embodiments the cuff ring 719 can be formed of the same material and material thickness as the sheet of material 702 forming the tissue retrieval bag 700. In other embodiments, the cuff ring 719 can be formed of a different material and/or a material having a different thickness than the sheet of material 702.

With reference to FIGS. 37-38, in the illustrated embodiment, the cuff ring 719 is positioned adjacent the recess 713 formed in the sheet of material 702 between the side segments 715 of the cuff 712. The cuff ring 719 is joined to the sheet of material 702 such as by welding (FIG. 37) to form a cuff assembly comprising a pair of side segments 715 and a cuff ring 719. The cuff assembly is then folded to retain a cord loop 42 and joined to the sheet of material 702. (FIG. 38). The sheet of material can then be folded and joined as described with respect to the tissue retrieval bag of FIGS. 23-24.

With reference to FIGS. 39-40, an embodiment of tissue retrieval bag 700 substantially as discussed with respect to FIGS. 23-24 is illustrated. The tissue retrieval bag 700 of FIGS. 39-40 further comprises a cuff ring 719 extending between the distal ends of the side segments 715 of the cuff 712. Advantageously, the cuff ring 719 can reinforce the cuff 712 at the distal end thereof. The reinforcement increases the tensile strength of the tissue retrieval bag to allow more force to be applied during extraction of the cinched bag through the abdominal wall.

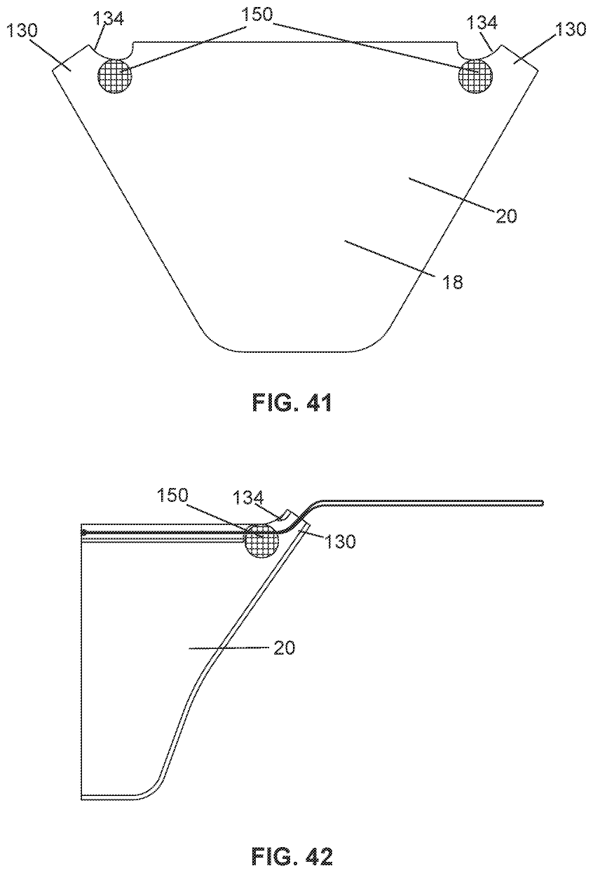

With reference to FIGS. 41-42, an embodiment of tissue retrieval bag is illustrated. In the illustrated embodiment, the tissue retrieval bag 20 of FIGS. 8-9 and 12-13 has been further reinforced with circular reinforcing tabs or reinforcing disks 150 joined to the sheet of material 18 thereof. In the illustrated embodiment, the reinforcing disks 150 are positioned on the sheet of material 18 adjacent the radiused upper edge 134 of the proximal upper portion 130, desirably providing additional strength adjacent a potential stress concentration in the tissue retrieval bag 20. In the illustrated embodiment, two reinforcing disks 150 are joined to the sheet of material, with each reinforcing disk 150 positioned adjacent an upper edge 134 at each side of the sheet of material 18 such that when the sheet of material is folded (FIG. 41) to form the tissue retrieval bag 20, the reinforcing disks 150 overlie one another. In other embodiments, a single reinforcing disk, positioned adjacent an upper edge on one side of the sheet of material 18 can be used.

With continued reference to FIGS. 41-42, in certain embodiments each reinforcing disk 150 comprise a generally circular portion of a sheet of material. In various embodiments, the reinforcing disk 150 can comprise the same material and material thickness as the sheet of material 18. In other embodiments, the reinforcing disk can comprise a different material and/or different thickness of material than the sheet of material 18 forming the tissue retrieval bag 20.

With continued reference to FIGS. 41-42, the illustrated embodiment of tissue retrieval bag 20 having reinforcing disks 150 can advantageously increase the strength of the bag during extraction of a closed bag for a redeployable system. Desirably, a circular reinforcement feature, such as the reinforcing disk 150 can facilitate manufacturing the tissue retrieval bag 20 as placement and welding during manufacturing can be simplified as compared with reinforcement features having more complex shapes. Moreover, unlike certain other reinforcement shapes, the same reinforcing disk 150 component can be used on either the left or right side of a sheet of material of a tissue retrieval bag pattern regardless as to whether the material is weldable on one or both sides. Thus, while materials that are weldable on both sides such as polyurethane, ripstop nylon, and polyurethane laminates may be preferable for tissue retrieval bag configurations having reinforced features with distinct left and right side reinforcement geometries, such material selection considerations would not apply to the illustrated circular reinforcing disks 150.

While certain embodiments of redeployable tissue retrieval bags have been described herein, it is contemplated that modifications to and further combinations of the aspects of these tissue retrieval bags can be made in still further embodiments of tissue retrieval bag within the scope of this application. For example, in certain further embodiments, any of the various reinforcements to the upper proximal portions of the tissue retrieval bags described with respect to FIGS. 14-19 and 41-42 can be added to the embodiments of tissue retrieval bags having an elongate profile described with respect to FIGS. 21-40. Furthermore, certain additional aspects described below can be incorporated into the embodiments herein in still other embodiments of tissue retrieval bags advantageously configured for redeployable operation.

In certain embodiments, the upper proximal portion of the retrieval bag can include an edge having a radius that is increased to a relatively large size to reduce the stresses in this area.

In various embodiments, after the bead is attached to the retrieval bag, during assembly the bead and the upper proximal portion of the retrieval bag can be rotated about the central longitudinal axis relative to the cuff and open end of the bag. In one embodiment, the bead and upper proximal portion can be rotated 180 degrees. In other embodiments, the bead and upper proximal portion are rotated an angular rotation more than or less than 180 degrees. In certain embodiments, the bead and upper proximal portion are rotated at least 90 degrees about the central longitudinal axis relative to the cuff and open end of the bag. During initial assembly of the tissue retrieval system, the support arms are inserted through the rotated bead and into the cuff of the retrieval bag to maintain the bead and the upper proximal portion in this orientation. This rotation results in a twisting of the upper proximal portion and can reduce the stresses in the upper radius resulting in a retrieval bag with an increased strength in this area.

In various embodiments, the bead and annular clamp can be sized and configured with an elongated profile to enable a retrieval bag with a lower radius in the upper proximal portion to be assembled with the lower radius fully encased within the bead and annular clamp. Such an encased positioning can desirably reduce the effects of the stress concentration at the lower radius and provide increased strength in this area.

Although this application discloses certain preferred embodiments and examples, it will be understood by those skilled in the art that the present inventions extend beyond the specifically disclosed embodiments to other alternative embodiments and/or uses of the inventions and obvious modifications and equivalents thereof. Further, the various features of these inventions can be used alone, or in combination with other features of these inventions other than as expressly described above. Thus, it is intended that the scope of the present inventions herein disclosed should not be limited by the particular disclosed embodiments described above, but should be determined only by a fair reading of the claims which follow.

What is claimed is:

1. A tissue retrieval system comprising:
a tubular introducer having a proximal end and a distal end and defining a central longitudinal axis between the proximal end and the distal end, the tubular introducer having a lumen extending between the proximal end and the distal end;
an actuator longitudinally slidable within the lumen of the introducer, the actuator having a proximal end and a distal end;
a pair of support arms extending distally along the central longitudinal axis from the distal end of the actuator;
a tissue retrieval bag removably coupled to the support arms, the tissue retrieval bag having an open end and a closed end opposite the open end, the tissue retrieval bag comprising:
a cuff extending longitudinally at the open end between a proximal end of the open end and a distal end of the open end, the cuff configured to receive the pair of support arms; and
an upper proximal portion positioned at the proximal end of the cuff and extending transversely to the longitudinal axis in a direction longitudinally proximally and away from the closed end; and
a bead coupled to the upper proximal portion, the bead configured to slidably receive the pair of support arms.

2. The tissue retrieval system of claim 1, wherein the tissue retrieval bag comprises a distal edge extending from the open end at the distal end of the cuff to the closed end.

3. The tissue retrieval system of claim 2, wherein the distal edge comprises a folded edge.

4. The tissue retrieval system of claim 1, wherein the tissue retrieval bag comprises a proximal edge extending from the open end at the proximal end of the cuff to the closed end.

5. The tissue retrieval system of claim 4, wherein the proximal edge comprises a welded seam.

6. The tissue retrieval system of claim 5, wherein the upper proximal portion comprises an upper edge and a lower edge opposite the upper edge.

7. The tissue retrieval system of claim 6, wherein the lower edge is colinear with the proximal edge.

8. The tissue retrieval system of claim 6, wherein the upper edge comprises a radiused profile.

9. The tissue retrieval system of claim 8, wherein the radiused profile has a first radiused portion having a first radius, a second radiused portion having a second radius, and a transition profile.

10. The tissue retrieval system of claim 9, wherein the first radius is larger than the second radius.

11. The tissue retrieval system of claim 1, wherein the tissue retrieval bag is operable between a stowed position in which the tissue retrieval bag is positioned within the introducer and a first deployed position in which the tissue retrieval bag is deployed from the introducer and coupled to the support arms such that longitudinal movement of the actuator within the lumen repeatably actuates the tissue retrieval bag between an open configuration and a closed configuration.

12. A tissue retrieval system comprising:
a tubular introducer having a proximal end and a distal end and defining a central longitudinal axis between the proximal end and the distal end, the tubular introducer having a lumen extending between the proximal end and the distal end;
an actuator longitudinally slidable within the lumen of the introducer, the actuator having a proximal end and a distal end;
a pair of support arms extending distally along the central longitudinal axis from the distal end of the actuator;
a tissue retrieval bag removably coupled to the support arms, the tissue retrieval bag having an open end and a closed end opposite the open end and defining a depth axis extending from the open end to the closed end orthogonally to the central longitudinal axis, wherein the open end extends along the central longitudinal axis from a proximal end to a distal end, the tissue retrieval bag comprising:
a distal edge extending from the distal end of the open end to the closed end;
a proximal edge extending from the proximal end of the open end to the closed end, the proximal edge extending transversely to the central longitudinal axis; and
an upper proximal portion positioned at the proximal end of the open end and extending in a direction colinear with the proximal edge away from the closed end with respect to the depth axis; and
a bead coupled to the upper proximal portion, the bead configured to slidably receive the pair of support arms.

13. The tissue retrieval system of claim 12, wherein the tissue retrieval bag is formed of a sheet of material.

14. The tissue retrieval system of claim 13, wherein the proximal edge comprises a welded seam.

15. The tissue retrieval system of claim 14, wherein the upper proximal portion comprises an upper edge and a lower edge opposite the upper edge.

16. The tissue retrieval system of claim 15, wherein the lower edge comprises a welded seam that is colinear with the proximal edge.

17. The tissue retrieval system of claim 15, wherein the upper edge comprises a radiused profile.

18. A tissue retrieval bag for use in a redeployable tissue retrieval system, the tissue retrieval bag comprising:
an open end extending along a central longitudinal axis from a proximal end of the open end to a distal end of the open end;
a closed end opposite the open end and defining a depth axis extending from the open end to the closed end orthogonally to the central longitudinal axis;
a distal edge extending from the distal end of the open end to the closed end;
a proximal edge extending from the proximal end of the open end to the closed end, the proximal edge extending transversely to the central longitudinal axis;
an attachment tab positioned at the proximal end of the open end and extending in a direction colinear with the proximal edge away from the closed end with respect to the depth axis; and
a bead coupled to the attachment tab.

19. The tissue retrieval bag of claim 18, wherein the attachment tab comprises a lower edge and an upper edge.

20. The tissue retrieval bag of claim 19, wherein the lower edge is colinear with the proximal edge.

21. The tissue retrieval bag of claim 19, wherein the upper edge comprises a radiused profile.
